(19) United States
(12) Patent Application Publication
(10) Pub. No.: US 2007/0238751 A1
(43) Pub. Date: Oct. 11, 2007

(54) Muscarinic Acetylcholine Receptor Antagonists

(76) Inventors: Dramane I. Laine, King of Prussia, PA (US); Michael R. Palovich, King of Prussia, PA (US); Haibo Xie, King of Prussia, PA (US); Noemie Buffet, Bourg-La-Reine (FR)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/599,717

(22) PCT Filed: Apr. 7, 2005
(86) PCT No.: PCT/US05/11975

§ 371 Date: Oct. 6, 2006

Related U.S. Application Data
(60) Provisional application No. 60/560,155, filed on Apr. 7, 2004.

(51) Int. Cl. A61K 31/439; A61P 11/00; C07D 221/22

(52) U.S. Cl. 514/299; 546/112

(57) ABSTRACT

Muscarinic Acetylcholine receptor antagonists and methods of using them are provided.

### FIELD OF THE INVENTION

This invention relates to the carbamate derivatives of 8-azoniabicyclo[3.2.1]octanes, pharmaceutical compositions, and use thereof in treating muscarinic acetylcholine receptor mediated diseases of the respiratory tract.

### BACKGROUND OF THE INVENTION

Acetylcholine released from cholinergic neurons in the peripheral and central nervous systems affects many different biological processes through interaction with two major classes of acetylcholine receptors—the nicotinic and the muscarinic acetylcholine receptors. Muscarinic acetylcholine receptors (mAChRs) belong to the superfamily of G-protein coupled receptors that have seven transmembrane domains. There are five subtypes of mAChRs, termed M₁-M₅, and each is the product of a distinct gene. Each of these five subtypes displays unique pharmacological properties. Muscarinic acetylcholine receptors are widely distributed in vertebrate organs where they mediate many of the vital functions. Muscarinic receptors can mediate both inhibitory and excitatory actions. For example, in smooth muscle found in the airways, M₃ mAChRs mediate contractile responses. For review, please see Caulfield (1993 Pharmac. Ther. 58:319-79).

In the lungs, mAChRs have been localized to smooth muscle in the trachea and bronchi, the submucosal glands, and the parasympathetic ganglia. Muscarinic receptor density is greatest in parasympathetic ganglia and then decreases in density from the submucosal glands to tracheal and then bronchial smooth muscle. Muscarinic receptors are nearly absent from the alveoli. For review of mACHR expression and function in the lungs, please see Fryer and Jacoby (1998 Am J Respir Crit Care Med 158(5, pt 3) S 154-60).

Three subtypes of mAChRs have been identified as important in the lungs, M₁, M₂ and M₃ mAChRs. The M₃ mAChRs, located on airway smooth muscle, mediate muscle contraction. Stimulation of M₃ mAChRs activates the enzyme phospholipase C via binding of the stimulatory G protein Gq/11 (Gs), leading to liberation of phosphatidyl inositol-4,5-bisphosphate, resulting in phosphorylation of contractile proteins. M₃ mAChRs are also found on pulmonary submucosal glands. Stimulation of this population of M₃ mAChRs results in mucus secretion.

M₂ mAChRs make up approximately 50-80% of the cholinergic receptor population on airway smooth muscles. Although the precise function is still unknown, they inhibit catecholaminergic relaxation of airway smooth muscle via inhibition of cAMP generation. Neuronal M₂ mAChRs are located on postganglionic parasympathetic nerves. Under normal physiologic conditions, neuronal M₂ mAChRs provide tight control of acetylcholine release from parasympathetic nerves. Inhibitory M₂ mAChRs have also been demonstrated on sympathetic nerves in the lungs of some species. These receptors inhibit release of noradrenaline, thus decreasing sympathetic input to the lungs.

M₁ mAChRs are found in the pulmonary parasympathetic ganglia where they function to enhance neurotransmission. These receptors have also been localized to the peripheral lung parenchyma, however their function in the parenchyma is unknown.

Muscarinic acetylcholine receptor dysfunction in the lungs has been noted in a variety of different pathophysiological states. In particular, in asthma and chronic obstructive pulmonary disease (COPD), inflammatory conditions lead to loss of inhibitory M₂ muscarinic acetylcholine autoreceptor function on parasympathetic nerves supplying the pulmonary smooth muscle, causing increased acetylcholine release following vagal nerve stimulation (Fryer et al. 1999 Life Sci 64 (6-7) 449-55). This mAChR dysfunction results in airway hyperreactivity and hyperresponsiveness mediated by increased stimulation of M₃ mAChRs. Thus the identification of potent mAChR antagonists would be useful as therapeutics in these mAChR-mediated disease states.

COPD is an imprecise term that encompasses a variety of progressive health problems including chronic bronchitis, chronic bronchiolitis and emphysema, and it is a major cause of mortality and morbidity in the world. Smoking is the major risk factor for the development of COPD; nearly 50 million people in the U.S. alone smoke cigarettes, and an estimated 3,000 people take up the habit daily. As a result, COPD is expected to rank among the top five as a world-wide health burden by the year 2020. Inhaled anti-cholinergic therapy is currently considered the “gold standard” as first line therapy for COPD (Pauwels et al. 2001 Am. J. Respir. Crit. Care Med. 163:1256-1276).

Despite the large body of evidence supporting the use of anti-cholinergic therapy for the treatment of airway hyperreactive diseases, relatively few anti-cholinergic compounds are available for use in the clinic for pulmonary indications. More specifically, in United States, Ipratropium Bromide (Atrovent©; and Combivent©, in combination with albuterol) is currently the only inhaled anti-cholinergic marketed for the treatment of airway hyperreactive diseases. While this compound is a potent anti-muscarinic agent, it is short acting, and thus must be administered as many as four times daily in order to provide relief for the COPD patient. In Europe and Asia, the long-acting anti-cholinergic Tiotropium Bromide (Spiriva©) was recently approved, however this product is currently not available in the United States. Thus, there remains a need for novel compounds that are capable of causing blockade at mAChRs which are long acting and can be administered once-daily for the treatment of airway hyperreactive diseases such as asthma and COPD. Since mAChRs are widely distributed throughout the body, the ability to apply anti-cholinergics locally and/or topically to the respiratory tract is particularly advantageous, as it would allow for lower doses of the drug to be utilized. Furthermore, the ability to design topically active drugs that have long duration of action, and in particular, are retained either at the receptor or by the lung, would allow the avoidance of unwanted side effects that may be seen with systemic anti-cholinergic use.

### SUMMARY OF THE INVENTION

This invention provides for a method of treating a muscarinic acetylcholine receptor (mAChR) mediated disease, wherein acetylcholine binds to an mAChR and which method comprises administering an effective amount of a compound of Formula (I) or a pharmaceutically acceptable salt thereof.

This invention also relates to a method of inhibiting the binding of acetylcholine to its receptors in a mammal in need thereof which comprises administering to aforementioned mammal an effective amount of a compound of Formula (I).

The present invention also provides for the novel compounds of Formula (I), and pharmaceutical compositions comprising a compound of Formula (I), and a pharmaceutical carrier or diluent.

Compounds of Formula (I) useful in the present invention are represented by the structure: 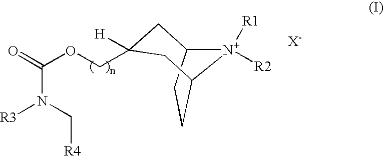
wherein:
R1 is a bond, hydrogen or C1-4 alkyl;
R2 is selected from the group consisting of hydrogen, C1-10 alkyl, halosubstitued C1-10 alkyl, C1-10 alkyl cyano, C2-10 alkenyl, cycloalkyl, C1-10 alkylcycloalkyl, cycloakyl C1-10 alkyl, and (CR8R8)q-ORa;
R3 and R4 are independently selected from the group consisting of 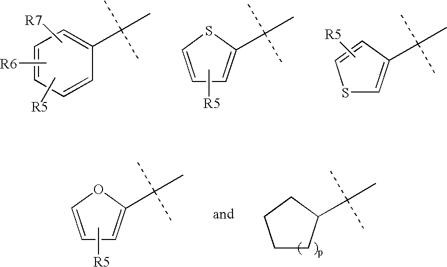
wherein R5, R6 and R7 are, independently, selected from the group consisting of hydrogen, halogen, C1-4 alkyl, C2-5 alkenyl, C1-4 alkoxy, halosubstituted C1-4 alkoxy, halosubstituted C1-4 alkyl, hydroxy, and cyano;
n is an integer having a value of 0 to 2;
p is an integer having a value of 0 to 3;
q is an integer having a value of 2 to 10;
Ra is selected from the group consisting of hydrogen, C1-10 alkyl, aryl, aryl C1-10 alkyl, C1-4 alkyl aryl, halosubstituted C1-10 alkyl, C1-10 alkoxy, halosubstituted C1-10 alkoxy, C1-10 alkyl cyano and C2-10 alkenyl;
R8 is hydrogen, halogen or C1-4 alkyl; and
X— is a physiologically acceptable anion, such as chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate.

### DETAILED DESCRIPTION OF THE INVENTION

This invention related to novel 8-azoniabicyclo[3.2.1]octane carbamate compounds, pharmaceutical compositions, processes for their preparation, and use thereof in treating mAChR mediated diseases.

In a preferred embodiment of the present invention the compound is of formula (I) herein below: 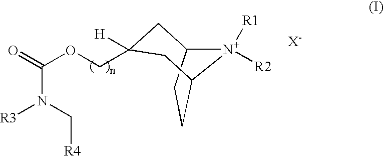
wherein:
R1 is a bond, hydrogen or C1-4 alkyl
R2 is selected from the group consisting of hydrogen, C1-4 alkyl, C2-5 alkenyl, C1-4 alkylcycloalkyl, and (CR8R8)q-ORa;
R3 and R4 are independently selected from the group consisting of 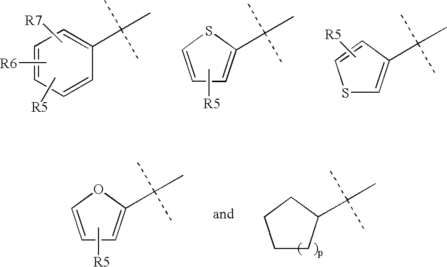
wherein R5, R6 and R7 are independently selected from the group consisting of hydrogen, halogen, C1-4 alkyl, C2-5 alkenyl, C1-4 alkoxy, and cyano;
n is an integer having a value of 0 or 1;
p is an integer having a value of 1 or 2;
q is an integer having a value of 2 to 4;
Ra is selected from the group consisting of hydrogen, C1-4 alkyl, aryl, aryl C1-4 alkyl, C1-4 alkyl aryl, and C1-10 alkyl;
R8 is hydrogen; and
X— is a physiologically acceptable anion, such as chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate.

All of the aryl, heteroaryl, and heterocyclic containing moieties may be optionally substituted as defined herein below.

For use herein the term “the aryl, heteroaryl, and heterocyclic containing moieties” refers to both the ring and the alkyl, or if included, the alkenyl rings, such as aryl, arylallkyl, and aryl alkenyl rings. The term “moieties” and “rings” may be interchangeably used throughout.

As used herein, “optionally substituted” unless specifically defined shall mean such groups as halogen, such as fluorine, chlorine, bromine or iodine; hydroxy; hydroxy substituted C₁₋₁₀alkyl; C₁₋₁₀ alkoxy, such as methoxy or ethoxy; S(O)_{m′}C₁₋₁₀ alkyl, wherein m′ is 0, 1 or 2, such as methyl thio, methyl sulfinyl or methyl sulfonyl; amino, mono & di-substituted amino, such as in the NR₁₀R₁₁ group; NHC(O)R₉; C(O)NR₁₀R₁₁; C(O)OH; S(O)₂NR₁₀R₁₁; NHS(O)₂R₉, C₁₋₁₀ alkyl, such as methyl, ethyl, propyl, isopropyl, or t-butyl; halosubstituted C₁₋₁₀ alkyl, such CF₃; an optionally substituted aryl, such as phenyl, or an optionally substituted arylalkyl, such as benzyl or phenethyl, optionally substituted heterocylic, optionally substituted heterocyclicalkyl, optionally substituted heteroaryl, optionally substituted heteroaryl alkyl, wherein these aryl, heteroaryl, or heterocyclic moieties may be substituted one to two times by halogen; hydroxy; hydroxy substituted alkyl; C₁₋₁₀ alkoxy; S(O)_{m′}C₁₋₁₀ alkyl; amino, mono & di-substituted alkyl amino, such as in the NR₁₀R₁₁ group; C₁₋₁₀ alkyl, or halosubstituted C₁₋₁₀ alkyl, such as CF₃.

The following terms, as used herein, refer to:

“halo”—all halogens, that is chloro, fluoro, bromo and iodo.
“C₁₋₁₀alkyl” or “alkyl”—both straight and branched chain moieties of 1 to 10 carbon atoms, unless the chain length is otherwise limited, including, but not limited to, methyl, ethyl, n-propyl, iso-propyl, n-butyl, sec-butyl, iso-butyl, tert-butyl, n-pentyl and the like.
“cycloalkyl” is used herein to mean cyclic moiety, preferably of 3 to 8 carbons, including but not limited to cyclopropyl, cyclopentyl, cyclohexyl, and the like.
“alkenyl” is used herein at all occurrences to mean straight or branched chain moiety of 2-10 carbon atoms, unless the chain length is limited thereto, including, but not limited to ethenyl, 1-propenyl, 2-propenyl, 2-methyl-1-propenyl, 1-butenyl, 2-butenyl and the like.
“aryl”—phenyl and naphthyl;
“heteroaryl” (on its own or in any combination, such as “heteroaryloxy”, or “heteroaryl alkyl”)—a 5-10 membered aromatic ring system in which one or more rings contain one or more heteroatoms selected from the group consisting of N, O or S, such as, but not limited, to pyrrole, pyrazole, furan, thiophene, quinoline, isoquinoline, quinazolinyl, pyridine, pyrimidine, oxazole, tetrazole, thiazole, thiadiazole, triazole, imidazole, or benzimidazole.
“heterocyclic” (on its own or in any combination, such as “heterocyclicalkyl”)—a saturated or partially unsaturated 4-10 membered ring system in which one or more rings contain one or more heteroatoms selected from the group consisting of N, O, or S; such as, but not limited to, pyrrolidine, piperidine, piperazine, morpholine, tetrahydropyran, thiomorpholine, or imidazolidine. Furthermore, sulfur may be optionally oxidized to the sulfone or the sulfoxide.
“arylalkyl” or “heteroarylalkyl” or “heterocyclicalkyl” is used herein to mean C₁₋₁₀ alkyl, as defined above, attached to an aryl, heteroaryl or heterocyclic moiety, as also defined herein, unless otherwise indicated.
“sulfinyl”—the oxide S(O) of the corresponding sulfide, the term “thio” refers to the sulfide, and the term “sulfonyl” refers to the fully oxidized S(O)₂ moiety.
“wherein two R₁ moieties (or two Y moieties) may together form a 5 or 6 membered saturated or unsaturated ring” is used herein to mean the formation of an aromatic ring system, such as naphthalene, or is a phenyl moiety having attached a 6 membered partially saturated or unsaturated ring such as a C₆ cycloalkenyl, i.e. hexene, or a C₅ cycloalkenyl moiety, such as cyclopentene.
   Illustrative compounds of Formula (I) include:
(3-endo)-8,8-dimethyl-3-({[3-thienyl(3-thienylmethyl)amino]carbonyl}oxy)-8-azoniabicyclo[3.2.1]octane bromide;
(3-endo)-8,8-dimethyl-3-({[(phenylmethyl)(3-thienyl)amino]carbonyl}oxy)-8-azoniabicyclo[3.2.1]octane bromide;
(3-endo)-3-({[[(3-fluorophenyl)methyl](3-thienyl)amino]carbonyl}oxy)-8,8-dimethyl-8-azoniabicyclo[3.2.1]octane bromide;
(3-endo)-8,8-dimethyl-3-({[phenyl(2-thienylmethyl)amino]carbonyl}oxy)-8-azoniabicyclo[3.2.1]octane bromide;
(3-endo)-3-({[[(4-fluorophenyl)methyl](3-thienyl)amino]carbonyl}oxy)-8,8-dimethyl-8-azoniabicyclo[3.2.1]octane bromide;
(3-endo)-8,8-dimethyl-3-({[phenyl(3-thienylmethyl)amino]carbonyl}oxy)-8-azoniabicyclo[3.2.1]octane bromide;
(3-endo)-8,8-dimethyl-3-({[(phenylmethyl)(2-thienyl)amino]carbonyl}oxy)-8-azoniabicyclo[3.2.1]octane bromide;
(3-endo)-3-({[[(4-fluorophenyl)methyl](2-thienyl)amino]carbonyl}oxy)-8,8-dimethyl-8-azoniabicyclo[3.2.1]octane bromide;
(3-endo)-3-({[[(2-fluorophenyl)methyl](3-thienyl)amino]carbonyl}oxy)-8,8-dimethyl-8-azoniabicyclo[3.2.1]octane bromide;
(3-endo)-3-({[[(3-fluorophenyl)methyl](2-thienyl)amino]carbonyl}oxy)-8,8-dimethyl-8-azoniabicyclo[3.2.1]octane bromide,
(3-endo)-3-({[[(3-fluorophenyl)methyl](phenyl)amino]carbonyl}oxy)-8,8-dimethyl-8-azoniabicyclo[3.2.1]octane bromide;
(3-endo)-3-({[[(2-fluorophenyl)methyl](2-thienyl)amino]carbonyl}oxy)-8,8-dimethyl-8-azoniabicyclo[3.2.1]octane bromide;
(3-endo)-8,8-dimethyl-3-({[phenyl(phenylmethyl)amino]carbonyl}oxy)-8-azoniabicyclo[3.2.1]octane bromide;
(3-endo)-3-({[[(2,4-difluorophenyl)methyl](2-thienyl)amino]carbonyl}oxy)-8,8-dimethyl-8-azoniabicyclo[3.2.1]octane bromide;
(3-endo)-8,8-dimethyl-3-({[2-thienyl(3-thienylmethyl)amino]carbonyl}oxy)-8-azoniabicyclo[3.2.1]octane iodide;
(3-endo)-8-azabicyclo[3.2.1]oct-3-yl 2-thienyl(3-thienylmethyl)carbamate trifluoroacetate;
(3-endo)-8-azabicyclo[3.2.1]oct-3-yl 3-thienyl(3-thienylmethyl)carbamate trifluoroacetate;
(3-endo)-8,8-dimethyl-3-({[[(5-methyl-2-thienyl)methyl](phenyl)amino]carbonyl}oxy)-8-azoniabicyclo[3.2.1]octane bromide;
(3-endo)-8-azabicyclo[3.2.1]oct-3-ylmethyl (phenylmethyl)2-thienylcarbamate;
(3-endo)-8-methyl-8-azabicyclo[3.2.1]oct-3-yl 3-thienyl(3-thienylmethyl)carbamate;
(3-endo)-8-methyl-8-azabicyclo[3.2.1]oct-3-yl [(4-fluorophenyl)methyl]3-thienylcarbamate;
(3-endo)-8-methyl-8-azabicyclo[3.2.1]oct-3-yl (phenylmethyl)3-thienylcarbamate;
(3-endo)-3-({[(cyclohexylmethyl)(3-thienyl)amino]carbonyl}oxy)-8,8-dimethyl-8-azoniabicyclo[3.2.1]octane bromide;
(3-endo)-8-azabicyclo[3.2.1]oct-3-ylmethyl[(4-fluorophenyl)methyl]2-thienylcarbamate; (3-endo)-8-azabicyclo[3.2.1]oct-3-ylmethyl[(3,4-difluorophenyl)methyl]2-thienylcarbamate;
(3-endo)-8-(6-hydroxyhexyl)-8-methyl-3-({[3-thienyl(3-thienylmethyl)amino]carbonyl}oxy)-8-azoniabicyclo[3.2.1]octane bromide;
(3-endo)-8-azabicyclo[3.2.1]oct-3-ylmethyl[(2-fluorophenyl)methyl]3-thienylcarbamate;
(3-endo)-3-({[[(2-fluorophenyl)methyl](phenyl)amino]carbonyl}oxy)-8,8-dimethyl-8-azoniabicyclo[3.2.1]octane bromide;
(3-endo)-8-azabicyclo[3.2.1]oct-3-ylmethyl[(3-fluorophenyl)methyl]2-thienylcarbamate;
(3-endo)-8-azabicyclo[3.2.1]oct-3-ylmethyl[(3-fluorophenyl)methyl]3-thienylcarbamate;
(3-endo)-8-azabicyclo[3.2.1]oct-3-ylmethyl[(3,5-difluorophenyl)methyl]3-thienylcarbamate;
(3-endo)-8-azabicyclo[3.2.1]oct-3-ylmethyl 3-thienyl[(2,4,5-trifluorophenyl)methyl]carbamate;
(3-endo)-8-methyl-8-azabicyclo[3.2.1]oct-3-yl [(3-fluorophenyl)methyl]3-thienylcarbamate;
(3-endo)-8-azabicyclo[3.2.1]oct-3-ylmethyl[(2-fluorophenyl)methyl]2-thienylcarbamate;
(3-endo)-3-({[(3-furanylmethyl)(phenyl)amino]carbonyl}oxy)-8,8-dimethyl-8-azoniabicyclo[3.2.1]octane bromide;
(3-endo)-8-azabicyclo[3.2.1]oct-3-ylmethyl[(2,4-difluorophenyl)methyl]2-thienylcarbamate;
(3-endo)-8-azabicyclo[3.2.1]oct-3-ylmethyl[(2,3-difluorophenyl)methyl]3-thienylcarbamate;
(3-endo)-8-azabicyclo[3.2.1]oct-3-ylmethyl[(2,5-difluorophenyl)methyl]2-thienylcarbamate;
(3-endo)-8-azabicyclo[3.2.1]oct-3-ylmethyl phenyl(phenylmethyl)carbamate;
(3-endo)-8-methyl-8-azabicyclo[3.2.1]oct-3-yl phenyl(2-thienylmethyl)carbamate;
(3-endo)-8-methyl-8-azabicyclo[3.2.1]oct-3-yl [(2-fluorophenyl)methyl]3-thienylcarbamate;
(3-endo)-8,8-dimethyl-3-({[[(3-methyl-2-thienyl)methyl](phenyl)amino]carbonyl}oxy)-8-azoniabicyclo[3.2.1]octane bromide;
(3-endo)-8-azabicyclo[3.2.1]oct-3-ylmethyl 2-thienyl[(2,3,4-trifluorophenyl)methyl]carbamate;
(3-endo)-8-methyl-8-azabicyclo[3.2.1]oct-3-yl (phenylmethyl)2-thienylcarbamate;
(3-endo)-8-methyl-8-azabicyclo[3.2.1]oct-3-yl [(3-fluorophenyl)methyl]2-thienylcarbamate;
(3-endo)-8-methyl-8-azabicyclo[3.2.1]oct-3-yl phenyl(3-thienylmethyl)carbamate;
(3-endo)-8-methyl-8-azabicyclo[3.2.1]oct-3-yl [(4-fluorophenyl)methyl]2-thienylcarbamate;
(3-endo)-8-(6-hydroxyhexyl)-8-azabicyclo[3.2.1]oct-3-yl 3-thienyl(3-thienylmethyl)carbamate;
(3-endo)-8-methyl-8-azabicyclo[3.2.1]oct-3-yl [(2-fluorophenyl)methyl]2-thienylcarbamate;
(3-endo)-3-({[[(4-bromophenyl)methyl](3-thienyl)amino]carbonyl}oxy)-8,8-dimethyl-8-azoniabicyclo[3.2.1]octane bromide; and
(3-endo)-8,8-dimethyl-3-({[[(5-methyl-2-furanyl)methyl](phenyl)amino]carbonyl}oxy)-8-azoniabicyclo[3.2.1]octane bromide.
   Preferred compounds useful in the present invention include:
(3-endo)-8,8-dimethyl-3-({[3-thienyl(3-thienylmethyl)amino]carbonyl}oxy)-8-azoniabicyclo[3.2.1]octane bromide;
(3-endo)-8,8-dimethyl-3-({[(phenylmethyl)(3-thienyl)amino]carbonyl}oxy)-8-azoniabicyclo[3.2.1]octane bromide;
(3-endo)-3-({[[(3-fluorophenyl)methyl](3-thienyl)amino]carbonyl}oxy)-8,8-dimethyl-8-azoniabicyclo[3.2.1]octane bromide;
(3-endo)-8,8-dimethyl-3-({[phenyl(2-thienylmethyl)amino]carbonyl}oxy)-8-azoniabicyclo[3.2.1]octane bromide;
(3-endo)-3-({[[(4-fluorophenyl)methyl](3-thienyl)amino]carbonyl}oxy)-8,8-dimethyl-8-azoniabicyclo[3.2.1]octane bromide;
(3-endo)-8,8-dimethyl-3-({[phenyl(3-thienylmethyl)amino]carbonyl}oxy)-8-azoniabicyclo[3.2.1]octane bromide;
(3-endo)-8,8-dimethyl-3-({[(phenylmethyl)(2-thienyl)amino]carbonyl}oxy)-8-azoniabicyclo[3.2.1]octane bromide;
(3-endo)-3-({[[(4-fluorophenyl)methyl](2-thienyl)amino]carbonyl}oxy)-8,8-dimethyl-8-azoniabicyclo[3.2.1]octane bromide;
(3-endo)-3-({[[(2-fluorophenyl)methyl](3-thienyl)amino]carbonyl}oxy)-8,8-dimethyl-8-azoniabicyclo[3.2.1]octane bromide;
(3-endo)-3-({[[(3-fluorophenyl)methyl](2-thienyl)amino]carbonyl}oxy)-8,8-dimethyl-8-azoniabicyclo[3.2.1]octane bromide;
(3-endo)-3-({[[(3-fluorophenyl)methyl](phenyl)amino]carbonyl}oxy)-8,8-dimethyl-8-azoniabicyclo[3.2.1]octane bromide;
(3-endo)-3-({[[(2-fluorophenyl)methyl](2-thienyl)amino]carbonyl}oxy)-8,8-dimethyl-8-azoniabicyclo[3.2.1]octane bromide;
(3-endo)-8,8-dimethyl-3-({[phenyl(phenylmethyl)amino]carbonyl}oxy)-8-azoniabicyclo[3.2.1]octane bromide;
(3-endo)-3-({[[(2,4-difluorophenyl)methyl](2-thienyl)amino]carbonyl}oxy)-8,8-dimethyl-8-azoniabicyclo[3.2.1]octane bromide;
(3-endo)-8,8-dimethyl-3-({[2-thienyl(3-thienylmethyl)amino]carbonyl}oxy)-8-azoniabicyclo[3.2.1]octane iodide;
(3-endo)-8-azabicyclo[3.2.1]oct-3-yl 2-thienyl(3-thienylmethyl)carbamate trifluoroacetate; and
(3-endo)-8-azabicyclo[3.2.1]oct-3-yl 3-thienyl(3-thienylmethyl)carbamate trifluoroacetate.

### Methods of Preparation

The compounds of Formula (I) may be obtained by applying synthetic procedures, some of which are illustrated in the Schemes below. The synthesis provided for these Schemes is applicable for producing compounds of Formula (I) having a variety of different R1, R2 R3 and R4 groups which are reacted, employing substituents which are suitable protected, to achieve compatibility with the reactions outlined herein. Subsequent deprotection, in those cases, then affords compounds of the nature generally disclosed. While the Schemes are shown with compounds only of Formula (I), this is merely for illustration purpose only.

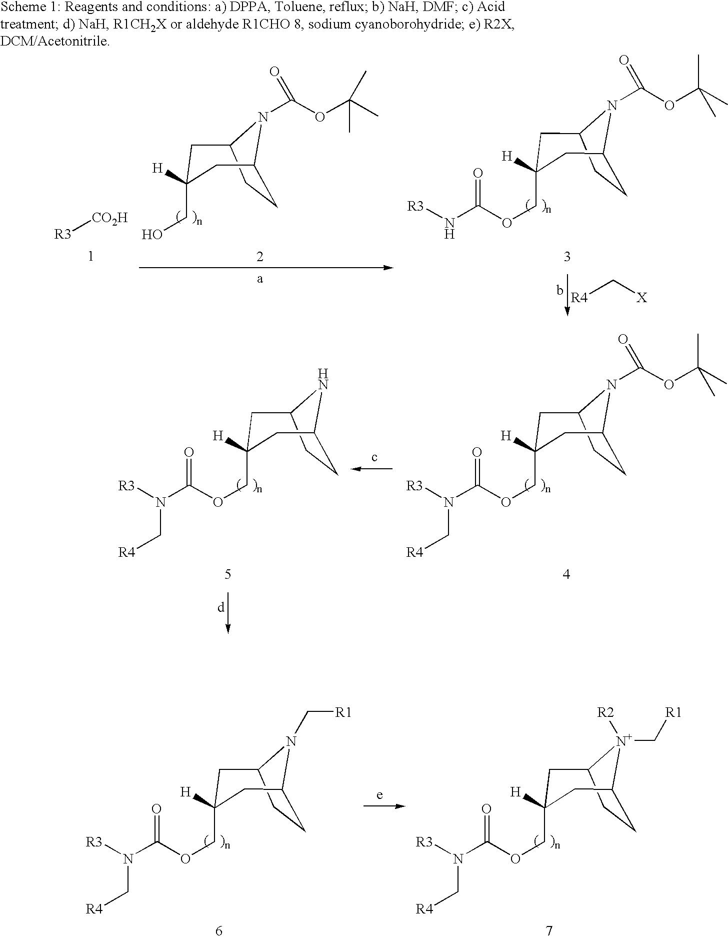

As outlined in Scheme 1, the desired compounds of Formula (I) can be prepared from a suitable carboxylic or aryl acid 1 in 3 to 5 steps. Firstly, the Curtius reaction between compound 1 and a suitably protected [3.2.1]bicyclic alcohol 2 using standard reagents well known in the art such as the commercially available diphenylphosphoryl azide (DPPA) reagent gives the carbamate intermediate 3. N-alkylation of compound 3 with a suitable alkylating agent such as an alkyl halide using standard reaction conditions such as sodium hydride in DMF gives the corresponding alkylated carbamate 4. Removal of the BOC protecting group of 4 using standard conditions such as treatment with p-toluenesulfonic acid in acetonitrile or trifluoroacetic acid in dichloromethane gives the compound 5 of Formula (I) (R1=R2=H). Further functionalisation of 5 can be carried out by alkyalting the molecule with a suitable alkyalting agent such as an alkyl bromide or via a reductive amination reaction with aldehyde 8 using suitable reagents well known in the art such as the commercially available solid supported cyanoborohydride resin to gives the tertiary amine 6 of Formula (I) (R1=H, R2 not H). Subsequent reaction of 6 with another alkylating agent R2X affords the related quaternary ammonium salt 7 of Formula (I) (R1 and R2 not H).

The required [3.2.1]bicyclic alcohol 2 (n=1) is not commercially available but can be prepared from compound 9 which has been previously described in the literature (T. Momone et al, J.C.S. Perkin. Trans. 1, 9, 1997, 1307-14). As shown in Scheme 2, compound 9 can undergo the Wittig reaction using standard reagents such as methyltriphenyl phosphonium bromide and potassium tert-butoxide to give the intermediate alkene 10. Hydroboration of compound 10 with disiamylborane followed by oxidation produced the corresponding alcohol 11. Subsequent removal of the benzylic moiety of 11 under hydrogenation conditions followed by protection of the ring nitrogen with a BOC group using standard conditions such as treatment with di-tert-butyl dicarbonate in the presence of a base such as sodium hydroxide gave the desired alcohol 2 (n=1).

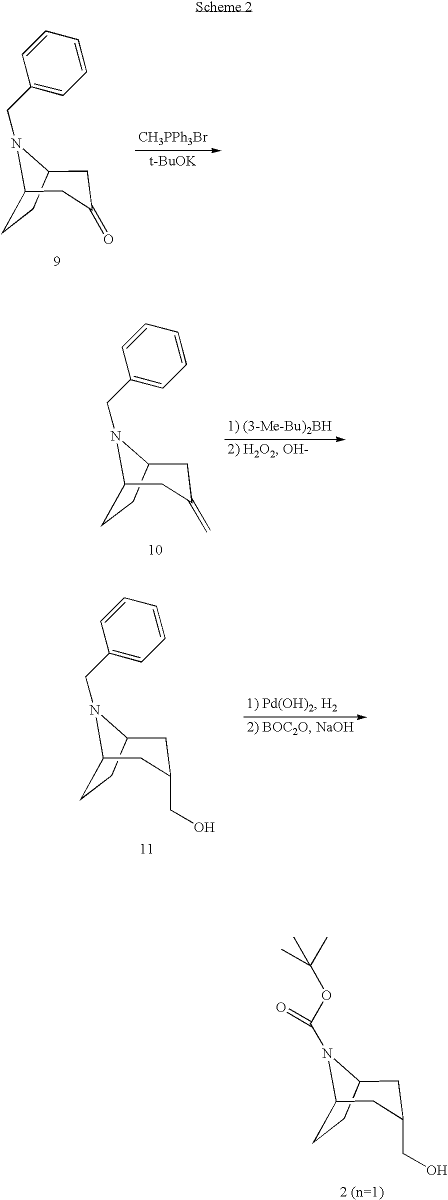

The required [3.2.1]bicyclic alcohol 2 (n=0) is not commercially available but can be prepared from the commercially available (3-endo)-8-methyl-8-azabicyclo[3.2.1]octan-3-ol 12 (a.k.a. tropine) using methods exemplified in the literature (Tetrahedron, 1998 (54), 10899-10914).

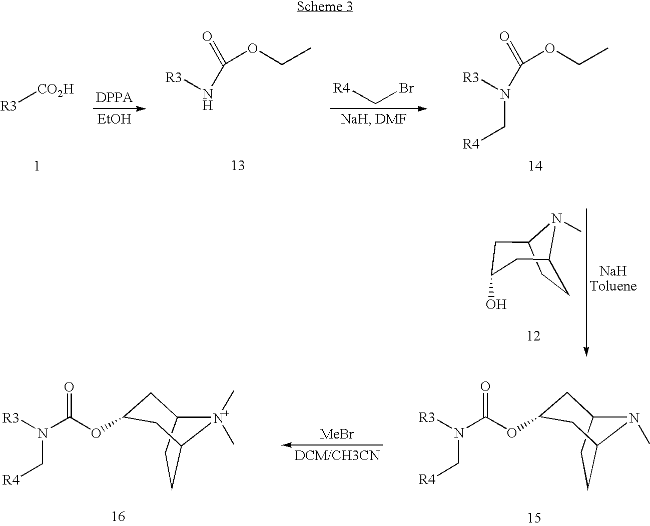

Alternatively, the desired compounds of Formula (I) can be prepared as outlined in Scheme 3. The Curtius reaction of a suitable carboxylic acid 1 with an alcohol such as ethanol using standard reagents well known in the art such as the commercially available diphenylphosphoryl azide (DPPA) reagent gives the corresponding ethyl carbamate 13. Alkylation of compound 13 with a suitable alkyl halide and using standard methods well known in the art such as sodium hydride in DMF produces the branched alkyl carbamate 14. These intermediates can be converted to the compounds 15 of Formula (I) (R1=CH₃, R2=nothing) by displacement of the alkoxy moiety with the commercially available compound 12, under standard basic conditions such sodium hydride in toluene. Subsequent reaction of 15 with another alkylating agent such as methyl bromide affords the related quaternary ammonium salt 16 of Formula (I) (R1 and R2=CH₃).

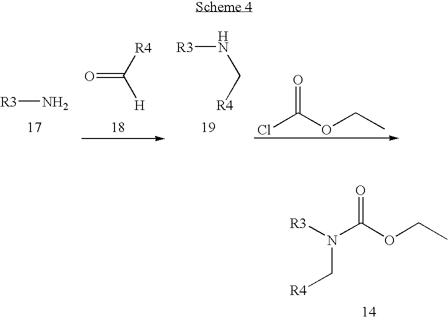

Alternatively, the carbamate derivatives 14 can be prepared from a suitable amine 17 as depicted in Scheme 4. The reductive amination reaction between amine 17 and an aldehyde 18 using suitable reagents well known in the art such as the commercially available solid supported cyanoborohydride resin gives the secondary amine 19. Subsequently, 19 can be reacted with the commercially available ethyl chloroformate under the appropriate basic conditions to give the suitable ethyl carbamate 14.

### SYNTHETIC EXAMPLES

The invention will now be described by reference to the following Examples which are merely illustrative and are not to be construed as a limitation of the scope of the present invention. Most reagents and intermediates are commercially available or are prepared according to procedures in the literature. The preparation of intermediates not described in the literature is also illustrated below.

Flash column chromatography was carried out using Merck 9385 silica unless stated otherwise.

LC/MS analyses were conducted under the following conditions:

| Column: | 3.3 cm × 4.6 mm ID, 3 um ABZ + PLUS | | |
|---|---|---|---|
| Flow Rate: | 3 ml/min | | |
| Injection | 5 μl | | |
| Volume: | | | |
| Temp: | Room temperature | | |
| Solvents: | A: 0.1% Formic Acid + 10 mMolar Ammonium Acetate. | | |
| | B: 95% Acetonitrile + 0.05% Formic Acid | | |
| Gradient: | Time | A % | B % |
| | 0.00 | 100 | 0 |
| | 0.70 | 100 | 0 |
| | 4.20 | 0 | 100 |
| | 5.30 | 0 | 100 |
| | 5.50 | 100 | 0 |

The Gilson preparatory HPLC was conducted under the following conditions:
Column: 75×33 mm I. D., S-5 um, 12 nm
Flow rate: 30 mL/min
Injection Volume: 0.800 mL
Room temperature
Solvent A: 0.1% trifluoroacetic acid in water
Solvent B: 0.1% trifluoroacetic acid in acetonitrile

### Preparation of 1,1-dimethylethyl-(3-endo)-(hydroxymethyl)-8-azabicyclo[3.2.1]octane-8-carboxylate

The compound was prepared in three steps:

### Step a: Preparation of 3-methylidene-8-(phenylmethyl)-8-azabicyclo[3.2.1]octane

A 500 ml flask with side arm, stirring bar, N₂ inlet, and septum stopper was charged with a solution of potassium tert-butoxide in THF (82 ml, 1M) and methyltriphenyl phosphonium bromide (29.2 g, 82 mmol). It was cooled to 0° C. under dry N₂, and anhydrous THF (100 ml) was added via syringe at 0° C. The yield solution was stirred for 20 min. 8-(Phenylmethyl)-8-azabicyclo[3.2.1]octan-3-one (14.0 g, 65 mmol) in anhydrous THF (40 ml) was added via syringe at 0° C. and the solution was stirred 1 hour at room temperature then quenched with water (6 ml). The mixture was acidified to pH 1 by addition of diluted aq. HCl and THF was removed in vacuo at 30° C. The residue was diluted with water (450 ml) and Ph₃PO was extracted with toluene (3×200 ml). The aqueous solution was basified with 6N NaOH (˜35 ml), and extracted with ethyl acetate (3×200 ml). The organic layers were combined, washed with saturated NaCl (3×100 ml), dried over Na₂SO₄, and evaporated to yield a crude product which was purified by flash chromatography (400 g of silica, ethyl acetate containing 0.1% TEA). 3-Methylidene-8-(phenylmethyl)-8-azabicyclo[3.2.1]octane was recovered as a yellow oil (11.3 g, 81.5%). LC/MS ESI R_{T} 1.27 min, MH⁺ 214. NMR (CDCl₃, 400 MHz; δ): 1.58 ppm (q, 2H), 1.80-2.05 ppm (m, 4H), 2.55 ppm (d, 2H), 3.28 ppm (s, 2H), 3.65 ppm (s, 2H), 4.80 ppm (s, 2H), 7.29 ppm (t, 1H), 7.35 ppm (t, 2H), 7.46 ppm (d, 2H).

### Step b: Preparation of (3-endo)-8-(phenylmethyl)-8-azabicyclo[3.2.1]oct-3-yl]methanol

A solution of disiamylborane was prepared by addition of 1.0 M borane in THF (20 ml, 20 mmol) to a 2.0 M solution of 2-methyl-2-butene in THF (20 ml, 40 mmol) at 0° C. under N₂. The solution was stirred 1 h at 0° C. before addition of 3-methylidene-8-(phenylmethyl)-8-azabicyclo[3.2.1]octane (1.07 g, 5 mmol) in 10 ml anhydrous THF. After stirring for 30 minutes at 0° C., the reaction mixture was warmed up to room temperature and allowed to stir overnight. The excess borane was quenched by careful addition of water (2 ml). The stirred solution was then oxidised at 0° C. by adding dropwise an aqueous solution of 30% H₂O₂ (3.87 ml, 45 mmol) over 30 minutes. The reaction mixture was neutralised with 3N HCl and the solvent was evaporated. The residue was taken up in ethyl acetate. Evaporation gave a viscous crude oil which was used directly for step c.

### Step c: Removal of the Benzyl Group and Protection with a BOC Group

A solution of (3-endo)-8-(phenylmethyl)-8-azabicyclo[3.2.1]oct-3-yl]methanol (1.16 g) (Schneider et al, Arch. Pharm., 1975, 308-365) in ethanol (20 ml) and 6N HCl (1 ml) containing palladium hydroxide on carbon (Pearlman's catalyst, 2.27 g, 22% (w/w)) was hydrogenated (55 psi H₂) at room temperature for 2 days. The catalyst was filtered off over Celite and the filtrate was evaporated under vacuum. The residue and di-tert-butyl dicarbonate (1.63 g, 7.5 mmol) were dissolved in 30 ml of dioxane: 1 N NaOH (2:1) and stirred for 14 hours at room temperature. The solvent was evaporated and the residue partitioned between ethyl acetate (3×25 ml) and water (25 ml). The combined organic layers were dried over Na₂SO₄ and evaporated. The residue oil was purified by flash chromatography (150 g of silica, hexane:ethyl acetate (1:1, containing 0.1% 2.0 M NH₃ in methanol)) to give the title compound as a colorless oil (0.65 g). LC/MS ESI R_{T} 1.65 min, MH⁺ 242. NMR (CDCl₃, 400 MHz; δ) 4.15 ppm (broad, 2H), 3.64 ppm (d, 2H), 2.20 ppm (broad, 2H), 1.97 ppm (broad, 2H), 1.85 ppm (m, 1H), 1.60 ppm (m, 2H), 1.40-1.50 ppm (s+broad, 11H).

### Intermediate 1: 1,1-dimethylethyl (3-endo)-({[(2-thienylamino)carbonyl]oxy}methyl)-8-azabicyclo[3.2.1]octane-8-carboxylate

To a stirred solution of 2-thiophenecarboxylic acid (128 mg, 1 mmol) in anhydrous THF (2 ml) was added dropwise triethyl amine (0.28 ml, 2 mmol), diphenylphosphoryl azide (300 mg, 1.1 mmol) and 1,1-dimethylethyl-(3-endo)-(hydroxymethyl)-8-azabicyclo[3.2.1]octane-8-carboxylate (361.5 mg, 1.5 mmol) at room temperature. The mixture was heated at reflux overnight under N₂. The solvent was evaporated and the residue was partitioned between ethyl acetate (30 ml) and water (15 ml). The organic phase was separated and the aqueous phase was further extracted with ethyl acetate (2×30 ml). The organic layers were combined, dried over Na₂SO₄, filtered and evaporated under vacuum to give a crude oil. Further purification of this material by Gilson HPLC, eluting with acetonitrile/water (10/90 to 90/10, v/v, over 10 min), gave 1,1-dimethylethyl (3-endo)-({[(2-thienylamino)carbonyl]oxy}methyl)-8-azabicyclo[3.2.1]octane-8-carboxylate (220 mg). LC/MS: m/z, 367 (M+H), 2.35 min.

### Intermediate 2: 1,1-dimethylethyl (3-endo)-({[(3-thienylamino)carbonyl]oxy}methyl)-8-azabicyclo[3.2.1]octane-8-carboxylate

Following the standard procedure outlined for Intermediate 1,3-thiophenecarboxylic acid (128 mg, 1 mmol) was reacted with 1,1-dimethylethyl-(3-endo)-(hydroxymethyl)-8-azabicyclo[3.2.1]octane-8-carboxylate (361.5 mg, 1.5 mmol) and diphenylphosphoryl azide (300 mg, 1.1 mmol) to give the title compound (277 mg, 74%). LC/MS: m/z, 367 (M+H), 2.33 min.

### Intermediate 3: 1,1-dimethylethyl (3-endo) 3-hydroxy-8-azabicyclo[3.2.1]octane-8-carboxylate

A stirred solution of (3-endo)-8-methyl-8-azabicyclo[3.2.1]octan-3-ol (also known as tropine, Aldrich cat. # T8-940-0) (10 g, 71 mmol) in 1,2-dichloroethane (150 ml) was cooled to 4° C., 1-chloroethyl chloroformate (38.1 ml, 352 mmol) was added dropwise and the solution was refluxed for 16 h. After cooling to room temperature, solvent was evaporated; the residue dissolved in MeOH and the solution was refluxed 3 h more. After cooling to room temperature, solvent was evaporated, the residue combined with t-BuOH (20 ml) and 6N NaOH (50 ml) and cooled to ˜15° C. A solution of di-tert-butyl dicarbonate (23.2 g, 107 mmol) in t-BuOH (35 ml) was added. The mixture was stirred at room temperature overnight. The mixture was diluted with EtOAc (500 ml), washed with water (200 ml), 5% citric acid (3×100 ml), water (100 ml) and saturated aqueous NaCl (100 ml), dried over Na₂SO₄ and concentrated. The residue was purified on a pad of silica gel (300 g) eluting with 2-4% methylene dichloride in MeOH to give a white solid (5.18 g, 32%. LC/MS: 228, 1.76 min.

### Intermediate 4: 1,1-dimethylethyl (3-endo) 3-{[(3-thienylamino)carbonyl]oxy}-8-azabicyclo[3.2.1]octane-8-carboxylate

Following the standard procedure outlined for Intermediate 1,3-thiophenecarboxylic acid (1.28 g, 10 mmol) was reacted with 1,1-dimethylethyl (3-endo) 3-{[(2-thienylamino)carbonyl]oxy}-8-azabicyclo[3.2.1]octane-8-carboxylate (Intermediate 3, 3.41 g, 15 mmol) to give the title compound (1.25 g, 36%). LC/MS: m/z, 353 (M+H), 2.25 min.

### Intermediate 5: 1,1-dimethylethyl (3-endo) 3-{[(2-thienylamino)carbonyl]oxy}-8-azabicyclo[3.2.1]octane-8-carboxylate

Following the standard procedure outlined for Intermediate 1,2-thiophenecarboxylic acid (0.64 g, 5 mmol) was reacted with 1,1-dimethylethyl (3-endo) 3-{[(2-thienylamino)carbonyl]oxy}-8-azabicyclo[3.2.1]octane-8-carboxylate (Intermediate 3, 1.71 g, 7.5 mmol) to give the title compound (1.27 g, 72%). LC/MS: m/z, 353 (M+H), 2.25 min.

### Example 1

### (3-endo)-8-azabicyclo[3.2.1]oct-3-ylmethyl (phenylmethyl)2-thienylcarbamate trifluoroacetate

To a solution of 1,1-dimethylethyl (3-endo)-({[(2-thienylamino)carbonyl]oxy}methyl)-8-azabicyclo[3.2.1]octane-8-carboxylate (220 mg, 0.6 mmol) in dry DMF (1 ml), 95% NaH was added at 0° C. The solution was stirred for 10 minutes and benzyl bromide (308 mg, 1.8 mmol) was added. The reaction mixture was allowed to warm to room temperature and stirred overnight. The combined organic layers were dried over Na₂SO₄ and evaporated under vacuum. The residual oil was mixed with 20% TFA in anhydrous dichloromethane (3 ml). The reaction mixture was stirred at room temperature for 30 min. then the solvent was removed under reduced pressure. The residue was purified by Gilson HPLC, eluting with acetonitrile/water/0.1% TFA (10/90 to 90/10, v/v, over 10 min) to give the title compound (254 mg, 90%). LC/MS: m/z, 357 (M+H), 1.53 min.

### Example 2

### (3-endo)-8,8-dimethyl-3-[({[(phenylmethyl)(2-thienyl)amino]carbonyl}oxy)methyl]-8-azoniabicyclo[3.2.1]octane iodide

(3-endo)-8-Azabicyclo[3.2.1]oct-3-ylmethyl (phenylmethyl)2-thienylcarbamate trifluorocaetate was dissolved in ethyl acetate (50 ml) and washed with saturated sodium bicarbonate (2×20 ml) and water (2×20 ml). The organic layer was dried over Na₂SO₄, filtered and evaporated. The residual solid was redissolved in methylene chloride (2 ml) and methanol (1 ml) then treated with methyl iodide (598 mg, 4.2 mmol) at room temperature followed by Na₂CO₃ (215 mg). The reaction mixture was filtered through a pad of celite after stirring overnight at room temperature to afford the title compound as a white powder (77.8 mg). LC/MS: m/z, 385 (M+H), 1.67 min.

### Example 3

### (3-endo)-8-azabicyclo[3.2.1]oct-3-ylmethyl{[4-(1,1-dimethylethyl)phenyl]methyl}2-thienylcarbamate trifluoroacetate

Following the standard procedure outlined in Example 1, 1,1-dimethylethyl (3-endo)-({[(2-thienylamino)carbonyl]oxy}methyl)-8-azabicyclo[3.2.1]octane-8-carboxylate (40 mg, 0.11 mmol) was reacted with 4-tert-butylbenzyl bromide (75 mg, 0.33 mmol) to give the title compound (26 mg, 57%). LC/MS: m/z, 413 (M+H), 2.08 min.

### Example 4

### (3-endo)-8-azabicyclo[3.2.1]oct-3-ylmethyl[(2-fluorophenyl)methyl]2-thienylcarbamate trifluoroacetate

Following the standard procedure outlined in Example 1, 1,1-dimethylethyl (3-endo)-({[(2-thienylamino)carbonyl]oxy}methyl)-8-azabicyclo[3.2.1]octane-8-carboxylate (40 mg, 0.11 mmol) was reacted with 2-fluorobenzyl bromide (62 mg, 0.33 mmol) to give the title compound (26 mg, 63%). LC/MS: m/z, 375 (M+H), 1.74 min.

### Example 5

### (3-endo)-8-azabicyclo[3.2.1]oct-3-ylmethyl[(3-fluorophenyl)methyl]2-thienylcarbamate trifluoroacetate

Following the standard procedure outlined in Example 1, 1,1-dimethylethyl (3-endo)-({[(2-thienylamino)carbonyl]oxy}methyl)-8-azabicyclo[3.2.1]octane-8-carboxylate (40 mg, 0.11 mmol) was reacted with 3-fluorobenzyl bromide (62 mg, 0.33 mmol) to give the title compound (12 mg, 31%). LC/MS: m/z, 375 (M+H), 1.78 min.

### Example 6

### (3-endo)-8-azabicyclo[3.2.1]oct-3-ylmethyl[(4-fluorophenyl)methyl]2-thienylcarbamate trifluoroacetate

Following the standard procedure outlined in Example 1, 1,1-dimethylethyl (3-endo)-({[(2-thienylamino)carbonyl]oxy}methyl)-8-azabicyclo[3.2.1]octane-8-carboxylate (40 mg, 0.11 mmol) was reacted with 4-fluorobenzyl bromide (62 mg, 0.33 mmol) to give the title compound (13 mg, 32%). LC/MS: m/z, 375 (M+H), 1.78 min.

### Example 7

### (3-endo)-8-azabicyclo[3.2.1]oct-3-ylmethyl[(4-cyanophenyl)methyl]2-thienylcarbamate trifluoroacetate

Following the standard procedure outlined in Example 1, 1,1-dimethylethyl (3-endo)-({[(2-thienylamino)carbonyl]oxy}methyl)-8-azabicyclo[3.2.1]octane-8-carboxylate (40 mg, 0.11 mmol) was reacted with 4-cyanobenzyl bromide (65 mg, 0.33 mmol) to give the title compound (28 mg, 67%). LC/MS: m/z, 382 (M+H), 1.63 min.

### Example 8

### (3-endo)-8-azabicyclo[3.2.1]oct-3-ylmethyl[(2,4-difluorophenyl)methyl]2-thienylcarbamate trifluoroacetate

Following the standard procedure outlined in Example 1, 1,1-dimethylethyl (3-endo)-({[(2-thienylamino)carbonyl]oxy}methyl)-8-azabicyclo[3.2.1]octane-8-carboxylate (40 mg, 0.11 mmol) was reacted with 2,4-difluorobenzyl bromide (62 mg, 0.33 mmol) to give the title compound (26 mg, 60%). LC/MS: m/z, 393 (M+H), 1.64 min.

### Example 9

### (3-endo)-8-azabicyclo[3.2.1]oct-3-ylmethyl[(2,5-difluorophenyl)methyl]2-thienylcarbamate trifluoroacetate

Following the standard procedure outlined in Example 1, 1,1-dimethylethyl (3-endo)-({[(2-thienylamino)carbonyl]oxy}methyl)-8-azabicyclo[3.2.1]octane-8-carboxylate (40 mg, 0.11 mmol) was reacted with 2,5-difluorobenzyl bromide (62 mg, 0.33 mmol) to give the title compound (28 mg, 65%). LC/MS: m/z, 393 (M+H), 1.63 min.

### Example 10

### (3-endo)-8-azabicyclo[3.2.1]oct-3-ylmethyl[(3,4-difluorophenyl)methyl]2-thienylcarbamate trifluoroacetate

Following the standard procedure outlined in Example 1, 1,1-dimethylethyl (3-endo)-({[(2-thienylamino)carbonyl]oxy}methyl)-8-azabicyclo[3.2.1]octane-8-carboxylate (40 mg, 0.11 mmol) was reacted with 3,4-difluorobenzyl bromide (62 mg, 0.33 mmol) to give the title compound (27 mg, 62%). LC/MS: m/z, 393 (M+H), 1.64 min.

### Example 11

### (3-endo)-8-azabicyclo[3.2.1]oct-3-ylmethyl 2-thienyl[(2,36-trifluorophenyl)methyl]carbamate trifluoroacetate

Following the standard procedure outlined in Example 1, 1,1-dimethylethyl (3-endo)-({[(2-thienylamino)carbonyl]oxy}methyl)-8-azabicyclo[3.2.1]octane-8-carboxylate (40 mg, 0.11 mmol) was reacted with 2,3,6-trifluoro-benzyl bromide (62 mg, 0.33 mmol) to give the title compound (29 mg, 64%). LC/MS: m/z, 411 (M+H), 1.64 min.

### Example 12

### (3-endo)-8-azabicyclo[3.2.1]oct-3-ylmethyl 2-thienyl[(2,3,4-trifluorophenyl)methyl]carbamate trifluoroacetate

Following the standard procedure outlined in Example 1, 1,1-dimethylethyl (3-endo)-({[(2-thienylamino)carbonyl]oxy}methyl)-8-azabicyclo[3.2.1]octane-8-carboxylate (40 mg, 0.11 mmol) was reacted with 2,3,4-trifluorobenzyl bromide (62 mg, 0.33 mmol) to give the title compound (45 mg, 99%). LC/MS: m/z, 411 (M+H), 1.59 min.

### Example 13

### (3-endo)-8-azabicyclo[3.2.1]oct-3-ylmethyl 2-thienyl[(2,4,6-trifluorophenyl)methyl]carbamate trifluoroacetate

Following the standard procedure outlined in Example 1, 1,1-dimethylethyl (3-endo)-({[(2-thienylamino)carbonyl]oxy}methyl)-8-azabicyclo[3.2.1]octane-8-carboxylate (40 mg, 0.11 mmol) was reacted with 2,4,6-trifluorobenzyl bromide (62 mg, 0.33 mmol) to give the title compound (20 mg, 44%). LC/MS: m/z, 411 (M+H), 1.68 min.

### Example 14

### (3-endo)-8-azabicyclo[3.2.1]oct-3-ylmethyl{[3,5-bis(methyloxy)phenyl]methyl}2-thienylcarbamate trifluoroacetate

Following the standard procedure outlined in Example 1, 1,1-dimethylethyl (3-endo)-({[(2-thienylamino)carbonyl]oxy}methyl)-8-azabicyclo[3.2.1]octane-8-carboxylate (220 mg, 0.60 mmol) was reacted with 3,5-dimethoxybenzyl bromide (277 mg, 1.12 mmol) to give the title compound (41 mg, 66%). LC/MS: m/z, 417 (M+H), 1.68 min.

### Example 15

### (3-endo)-8-azabicyclo[3.2.1]oct-3-ylmethyl{[3-(methyloxy)phenyl]methyl}2-thienylcarbamate trifluoroacetate

Following the standard procedure outlined in Example 1, 1,1-dimethylethyl (3-endo)-({[(2-thienylamino)carbonyl]oxy}methyl)-8-azabicyclo[3.2.1]octane-8-carboxylate (220 mg, 0.60 mmol) was reacted with 3-methoxybenzyl bromide (241 mg, 1.12 mmol) to give the title compound (155 mg, 62%). LC/MS: m/z, 387 (M+H), 1.74 min.

### Example 16

### (3-endo)-8-azabicyclo[3.2.1]oct-3-ylmethyl[(2-fluorophenyl)methyl]3-thienylcarbamate trifluorocaetate

Following the standard procedure outlined in Example 1, 1,1-dimethylethyl (3-endo)-({[(3-thienylamino)carbonyl]oxy}methyl)-8-azabicyclo[3.2.1]octane-8-carboxylate (40 mg, 0.11 mmol) was reacted with 2-fluorobenzyl bromide (62 mg, 0.33 mmol) to give the title compound (17 mg, 41%). LC/MS: m/z, 375 (M+H), 1.57 min.

### Example 17

### (3-endo)-8-azabicyclo[3.2.1]oct-3-ylmethyl[(3-fluorophenyl)methyl]3-thienylcarbamate trifluoroacetate

Following the standard procedure outlined in Example 1, 1,1-dimethylethyl (3-endo)-({[(3-thienylamino)carbonyl]oxy}methyl)-8-azabicyclo[3.2.1]octane-8-carboxylate (40 mg, 0.11 mmol) was reacted with 3-fluorobenzyl bromide (62 mg, 0.33 mmol) to give the title compound (40 mg, 97%). LC/MS: m/z, 375 (M+H), 1.62 min.

### Example 18

### (3-endo)-8-azabicyclo[3.2.1]oct-3-ylmethyl[(2,3-difluorophenyl)methyl]3-thienylcarbamate trifluoroacetate

Following the standard procedure outlined in Example 1, 1,1-dimethylethyl (3-endo)-({[(3-thienylamino)carbonyl]oxy}methyl)-8-azabicyclo[3.2.1]octane-8-carboxylate (40 mg, 0.11 mmol) was reacted with 2,3-difluorobenzyl bromide (62 mg, 0.33 mmol) to give the title compound (45 mg, 100%). LC/MS: m/z, 393 (M+H), 1.64 min.

### Example 19

### (3-endo)-8-azabicyclo[3.2.1]oct-3-ylmethyl[(3,5-difluorophenyl)methyl]3-thienylcarbamate trifluoroacetate

Following the standard procedure outlined in Example 1, 1,1-dimethylethyl (3-endo)-({[(3-thienylamino)carbonyl]oxy}methyl)-8-azabicyclo[3.2.1]octane-8-carboxylate (40 mg, 0.11 mmol) was reacted with 3,5-difluorobenzyl bromide (62 mg, 0.33 mmol) to give the title compound (50 mg, 100%). LC/MS: m/z, 393 (M+H), 1.64 min.

### Example 20

### (3-endo)-8-azabicyclo[3.2.1]oct-3-ylmethyl 3-thienyl[(2,3,6-trifluorophenyl)methyl]carbamate trifluoroacetate

Following the standard procedure outlined in Example 1, 1,1-dimethylethyl (3-endo)-({[(3-thienylamino)carbonyl]oxy}methyl)-8-azabicyclo[3.2.1]octane-8-carboxylate (40 mg, 0.11 mmol) was reacted with 2,3,6-trifluorobenzyl bromide (62 mg, 0.33 mmol) to give the title compound (64 mg, 100%). LC/MS: m/z, 411 (M+H), 1.60 min.

### Example 21

### (3-endo)-8-azabicyclo[3.2.1]oct-3-ylmethyl 3-thienyl[(2,4,5-trifluorophenyl)methyl]carbamate trifluoroacetate

Following the standard procedure outlined in Example 1, 1,1-dimethylethyl (3-endo)-({[(3-thienylamino)carbonyl]oxy}methyl)-8-azabicyclo[3.2.1]octane-8-carboxylate (40 mg, 0.11 mmol) was reacted with 2,4,5-trifluorobenzyl bromide (62 mg, 0.33 mmol) to give the title compound (58 mg, 100%). LC/MS: m/z, 411 (M+H), 1.65 min.

### Example 22

### (3-endo)-8-azabicyclo[3.2.1]oct-3-ylmethyl (3-methylphenyl)(phenylmethyl)carbamate trifluoroacetate

### 22-a: Preparation of 3-methyl-N-(phenylmethyl)aniline

Sodium triacetoxyborohydride (3.32 g, 15.6 mmol) was added to an ice-cooled solution of 3-methylaniline (398 mg, 3.7 mmol), benzaldehyde (472 mg, 4.5 mmol) and acetic acid (630 mg, 10.5 mmol) in methanol (15 ml). The reaction mixture was stirred at room temperature for 1 hour then quenched with saturated aq. NaHCO₃ (10 ml). Ethyl acetate (100 ml) was added, then the aqueous layer was separated and extracted with ethyl acetate (2×50 ml). The combined organic phases were dried over MgSO₄ and concentrated to afford 3-methyl-N-(phenylmethyl)aniline (350 mg, 47.5%). LC/MS: m/z, 198.2 (M+H), 1.82 min.

### 22-b Preparation of the Title Compound

A mixture of 1,1-dimethylethyl-(3-endo)-(hydroxymethyl)-8-azabicyclo[3.2.1]octane-8-carboxylate (750 mg, 3.1 mmol) and diisopropylethylamine (400 mg, 3.1 mmol) in dry THF (40 ml) was added dropwise to a solution of triphosgene (360 mg, 1.24 mmol) in dry THF (10 ml) at 0-5° C. under nitrogen. The mixture was stirred for 1.5 h, then a solution of 3-methyl-N-(phenylmethyl)aniline (99 mg, 0.51 mmol) in dry THF (10 ml) was added dropwise. The mixture was stirred for 16 h at room temperature. Water (10 ml) followed by ethyl acetate were added to the reaction. The organic phase was separated and the aqueous phase was further extracted with ethyl acetate. The combined organic layers were washed with brine, dried over Na₂SO₄, filtered and evaporated under vacuum to give a crude residue. Purification by Flash chromatography on silica eluting first with dichloromethane then with ethyl acetate gave the title compound as a pale yellow powder (358 mg). The solid was re-dissolved in dichloromethane (10 ml) and treated with TFA (4 ml) at room temperature. The reaction mixture was heated to 60° C. and stirred for half an hour then concentrated under vacuum. The residue was dissolved in DMSO (1.5 ml) and purified by Gilson preparatory HPLC, eluting with acetonitrile/water/O. 1% TFA (25/75, v/v to 85/15, v/v, over 10 min), to give the title compound (250 mg, 70%). LC/MS: m/z 365.4, (M+H), 1.68 min.

### Example 23

### (3-endo)-8-azabicyclo[3.2.1]oct-3-ylmethyl phenyl{[4-(trifluoromethyl)phenyl]methyl}carbamate trifluoroacetate

### 23-a Preparation of N-{[4-(trifluoromethyl)phenyl]methyl}aniline

Following the standard procedure outlined in Example 22a, aniline (346 mg, 3.7 mmol) was reacted with 4-(trifluoromethyl)benzaldehyde (776 mg, 4.5 mmol) in the presence of NaB(OAc)₃H (3.32 g, 15.6 mmol) and acetic acid (0.6 ml, 10.5 mmol) to give N-{[4-(trifluoromethyl)phenyl]methyl}aniline (495 mg). LC/MS: m/z, 252.2 (M+H), 2.37 min.

### 23-b Preparation of the Title Compound

Following the standard procedure outlined in Example 22b, 1,1-dimethylethyl-(3-endo)-(hydroxymethyl)-8-azabicyclo[3.2.1]octane-8-carboxylate (750 mg, 3.1 mmol) was reacted with N-{[4-(trifluoromethyl)phenyl]methyl}aniline (126 mg, 0.51 mmol) in the presence of triphosgene to give the title compound (216 mg, 62%). LC/MS: m/z, 419.2 (M+H), 1.73 min.

### Example 24

### (3-endo)-8-azabicyclo[3.2.1]oct-3-ylmethyl[(3,5-difluorophenyl)methyl](3-methylphenyl)carbamate trifluoroacetate

### 24-a N-[(3,5-difluorophenyl)methyl]-3-methylaniline

Following the standard procedure outlined in Example 22a, 3-methylaniline (398 mg, 3.72 mmol) was reacted with 3,5-difluorobenzaldehyde (633 mg, 4.5 mmol) in the presence of NaB(OAc)₃H (3.32 g, 15.6 mmol) and acetic acid (0.6 ml, 10.5 mmol) to give N-[(3,5-difluorophenyl)methyl]-3-methylaniline (578 mg). LC/MS: m/z, 233.8 (M+H), 2.33 min.

### 24-b Preparation of the Title Compound

Following the standard procedure outlined in Example 22b, 1,1-dimethylethyl-(3-endo)-(hydroxymethyl)-8-azabicyclo[3.2.1]octane-8-carboxylate (750 mg, 3.1 mmol) was reacted N-[(3,5-difluorophenyl)methyl]-3-methylaniline (116 mg, 0.51 mmol) in the presence of triphosgene to give the title compound (245 mg, 71%). LC/MS: m/z, 401.2 (M+H), 1.88 min.

### Example 25

### (3-endo)-8-azabicyclo[3.2.1]oct-3-ylmethyl (3-chlorophenyl) [(2,3-difluorophenyl)methyl]carbamate trifluoroacetate

### 25-a 3-chloro-N-[(2,3-difluorophenyl)methyl]aniline

Following the standard procedure outlined in Example 22a, 3-chloroaniline (472 mg, 3.7 mmol) was reacted with 2,3-difluorobenzaldehyde (633 mg, 0.46 mmol) in the presence of NaB(OAc)₃H (3.32 g, 15.6 mmol) and acetic acid (0.6 ml, 10.5 mmol) to give 3-chloro-N-[(2,3-difluorophenyl)methyl]aniline (610 mg, 65%). LC/MS: m/z, 254.2 (M+H), 2.57 min.

### 25-b Preparation of the Title Compound

Following the standard procedure outlined in Example 22b, 1,1-dimethylethyl-(3-endo)-(hydroxymethyl)-8-azabicyclo[3.2.1]octane-8-carboxylate (750 mg, 3.1 mmol) was reacted with 3-chloro-N-[(2,3-difluorophenyl)methyl]aniline (127 mg, 0.51 mmol) in the presence of triphosgene to give the title compound (295 mg, 73%). LC/MS: m/z, 421.4 (M+H), 1.92 min.

### Example 26

### (3-endo)-8-azabicyclo[3.2.1]oct-3-ylmethyl phenyl(phenylmethyl)carbamate trifluoroacetate

Following the standard procedure outlined in Example 22, 1,1-dimethylethyl-(3-endo)-(hydroxymethyl)-8-azabicyclo[3.2.1]octane-8-carboxylate (750 mg, 3.1 mmol) was reacted with N-(phenylmethyl)aniline (0.42 ml, 4.5 mmol) in the presence of triphosgene to give the title compound (108 mg, 21%). LC/MS: m/z, 351 (M+H).

### Example 27

### (3-endo)-3-[({[[(2-fluorophenyl)methyl](2-thienyl)amino]carbonyl}oxy)methyl]-8,8-dimethyl-8-azoniabicyclo[3.2.1]octane bromide

A solution of (3-endo)-8-azabicyclo[3.2.1]oct-3-ylmethyl[(2-fluorophenyl)methyl]2-thienylcarbamate trifluoroacetate (10.4 mg) in dichloromethane (0.1 ml) and acetonitrile (0.1 ml) was treated with sodium bicarbonate (5 mg) then with methyl bromide (0.1 ml of a 2M solution in tert-butylmethyl ether). After stirring at room temperature for 16 hours, the reaction mixture was filtered. The filtrate was evaporated under vacuum to afford the title compound (3.0 mg). LC/MS: m/z, 403 (M+), 1.84 min.

### Example 28

### (3-endo)-3-[({[[(3,5-difluorophenyl)methyl](3-thienyl)amino]carbonyl}oxy)methyl]-8,8-dimethyl-8-azoniabicyclo[3.2.1]octane bromide

Following the procedure outlined in Example 27, (3-endo)-8-azabicyclo[3.2.1]oct-3-ylmethyl[(3,5-difluorophenyl)methyl]3-thienylcarbamate trifluoroacetate was treated with methyl bromide to give the title compound (7.6 mg). LC/MS: m/z, 421.2 (M+), 1.81 min.

### Example 29

### (3-endo)-3-[({[[(3-fluorophenyl)methyl]3-thienyl)amino]carbonyl}oxy)methyl]-8,8-dimethyl-8-azoniabicyclo[3.2.1]octane bromide

Following the procedure outlined in Example 27, (3-endo)-8-azabicyclo[3.2.1]oct-3-ylmethyl[(3-fluorophenyl)methyl]3-thienylcarbamate trifluoroacetate was treated with methyl bromide to give the title compound (6.0 mg). LC/MS: m/z, 403.5.2 (M+), 1.78 mm.

### Example 30

### (3-endo)-3-[({[[(4-cyanophenyl)methyl](2-thienyl)amino]carbonyl}oxy)methyl]-8,8-dimethyl-8-azoniabicyclo[3.2.1]octane bromide

Following the procedure outlined in Example 27, (3-endo)-8-azabicyclo[3.2.1]oct-3-ylmethyl[(4-cyanophenyl)methyl]2-thienylcarbamate trifluoroacetate was treated with methyl bromide to give the title compound (4.0 mg). LC/MS: m/z, 410.4 (M+), 1.89 min.

### Example 31

### (3-endo)-3-[({[{[4-(1,1-dimethylethyl phenyl]methyl}(2-thienyl)amino]carbonyl}oxy)methyl]-8,8-dimethyl-8-azoniabicyclo[3.2.1]octane bromide

Following the procedure outlined in Example 27, (3-endo)-8-azabicyclo[3.2.1]oct-3-ylmethyl{[4-(1,1-dimethylethyl)phenyl]methyl}2-thienylcarbamate trifluoroacetate was treated with methyl bromide to give the title compound (4.6 mg). LC/MS: m/z, 441 (M+), 2.19 min.

### Example 32

### (3-endo)-8-methyl-8-azabicyclo[3.2.1]oct-3-yl (phenylmethyl)3-thienylcarbamate

### 32a: Ethyl 3-thienylcarbamate

Diphenyl phosphoryl azide (6.5 ml, 30.2 mmol) was added to mixture of 3-thienyl carboxylic (3 g, 23.5 mmol) and triethylamine (6.5 ml, 46.9 mmol) in ethanol (3 ml) and toluene (60 ml). The resulting mixture was allowed to stir at reflux for 12 hours. After cooling to room temperature, silica gel was added to the reaction mixture. The solvents were evaporated under vacuum and the resulting crude material absorbed onto silica was purified by flash chromatography eluting with a mixture ethyl acetate/hexane 1:9. Ethyl 3-thienylcarbamate was recovered as a white solid (2.4 g). LC/MS: m/z, 172 (M+), 1.61 min.

### 32b: Ethyl (phenylmethyl)3-thienylcarbamate

Sodium hydride (33 mg, 0.88 mmol) was added to solution of ethyl 3-thienylcarbamate (150 mg, 0.88 mmol) in DMF (2 ml). The resulting mixture was stirred at room temperature under argon for 30 minutes then added to a solution of benzyl bromide (0.13 ml, 1.14 mmol) in DMF (1 ml). After further stirring at room temperature under argon for 18 hours, the reaction mixture was quenched with water (0.2 ml) and the solvents were evaporated under vacuum to give an oily residue which was redissolved in DCM (2 ml) ad water (1 ml). The organic phase was separated and evaporated under vacuum to give ethyl (phenylmethyl)3-thienylcarbamate as a crude material (245 mg).

### 32.c (3-endo)-8-methyl-8-azabicyclo[3.2.1]oct-3-yl (phenylmethyl)3-thienylcarbamate

A solution of the commercially available (3-endo)-8-methyl-8-azabicyclo[3.2.1]octan-3-ol (216 mg, 1.5 mmol) in toluene (1.5 ml) was treated with sodium hydride (34 mg, 1.5 mmol) under argon for 30 minutes then added to the crude ethyl (phenylmethyl)3-thienylcarbamate (245 mg) previously prepared. The resulting mixture was stirred at reflux for 3 hours, then cooled to room temperature and quenched with 2 droplets of water. The solvent was evaporated under vacuum to give a crude material which was partitioned between DCM (10 ml) and water (5 ml). After separation of the two phases using a hydrophobic frit, the organic layer was loaded onto a 2 g aminopropyl SPE cartridge and eluted sequentially with toluene (17 ml), ethyl acetate (10 ml) and methanol (5 ml). The ethyl acetate fraction was evaporated under vacuum to afford the title compound as a white solid (35.5 mg). LC/MS m/z, 357.2 (M+), 1.77 min.

The compounds listed in Table 1 were prepared proceeding in a similar manner to Example 32, but replacing 3-thienyl carboxylic with the appropriate carboxylic acid for step a and replacing benzyl bromide with the appropriate alkyl halide for step b.

TABLE 1

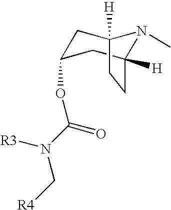

| Example | R3 | R4 | MS [M+] | Rt (min) |
|---|---|---|---|---|
| 33 | 3-thienyl | 4-fluoro-phenyl | 375 | 1.82 |
| 34 | 3-thienyl | 2-fluoro-phenyl | 375 | 1.71 |
| 35 | 3-thienyl | 3-fluoro-phenyl | 375 | 1.78 |
| 36 | 3-thienyl | 4-cyano-phenyl | 382 | 1.62 |
| 37 | 3-thienyl | 3-methoxy-phenyl | 387.4 | 1.67 |
| 38 | 3-thienyl | 2-bromo-phenyl | 435.2 | 1.84 |
| 39 | 3-thienyl | 3-bromo-phenyl | 435.2 | 1.86 |
| 40 | 3-thienyl | 4-bromo-phenyl | 435.2 | 1.84 |
| 41 | 3-thienyl | hexyl | 363.2 | 1.96 |
| 42 | phenyl | 2-fluoro-phenyl | 368.2 | 1.72 |
| 43 | phenyl | 3-fluoro-phenyl | 369.2 | 1.80 |
| 44 | phenyl | 4-cyano-phenyl | 376.2 | 1.61 |
| 45 | phenyl | 2-bromo-phenyl | 429.0 | 1.78 |
| 46 | phenyl | 4-bromo-phenyl | 429.0 | 1.85 |
| 47 | phenyl | 4-methoxy-phenyl | 381.2 | 1.64 |
| 48 | phenyl | phenyl | 351.0 | 1.73 |
| 49 | 2-thienyl | phenyl | 357.2 | 1.73 |
| 50 | 2-thienyl | 2-fluoro-phenyl | 375 | 1.69 |
| 51 | 2-thienyl | 3-fluoro-phenyl | 375 | 1.67 |
| 52 | 2-thienyl | 4-fluoro-phenyl | 375 | 1.74 |
| 53 | 2-thienyl | 2,4-difluorophenyl | 393.4 | 1.74 |
| 54 | 2-thienyl | 2-bromo-phenyl | 435.2 | 1.85 |
| 55 | 2-thienyl | 3-bromo-phenyl | 435.2 | 1.88 |
| 56 | 2-thienyl | 4-bromo-phenyl | 435.2 | 1.88 |
| 57 | 2-thienyl | 4-methoxy-phenyl | 387.4 | 1.70 |

### Example 58

### (3-endo)-8,8-dimethyl-3-({[(phenylmethyl)(3-thienyl)amino]carbonyl}oxy)-8-azoniabicyclo[3.2.1]octane bromide

Methyl bromide (0.8 ml of a 2.0 M in tert-butyl methyl ether, 1.6 mmol) was added to a solution of (3-endo)-8-methyl-8-azabicyclo[3.2.1]oct-3-yl (phenylmethyl)3-thienylcarbamate (60 mg, 0.17 mmol) in DCM (0.5 ml) and acetonitrile (0.5 ml). The resulting mixture was stirred at room temperature for 3 hours then evaporated under vacuum to give the title compound as a white solid (72 mg). LC/MS m/z, 371.2 (M+), 1.74 min.

The compounds listed in Table 2 were prepared proceeding in a similar manner to Example 58, but replacing (3-endo)-8-methyl-8-azabicyclo[3.2.1]oct-3-yl (phenylmethyl)3-thienylcarbamate with the appropriate tertiary amine.

TABLE 2

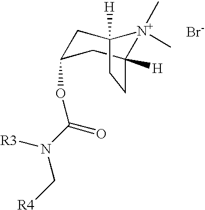

| Example | R3 | R4 | MS [M+] | Rt (min) |
|---|---|---|---|---|
| 59 | 3-thienyl | 4-fluoro-phenyl | 389.2 | 1.75 |
| 60 | 3-thienyl | 2-fluoro-phenyl | 389.2 | 1.70 |
| 61 | 3-thienyl | 3-fluoro-phenyl | 389.2 | 1.77 |
| 62 | 3-thienyl | 4-cyano-phenyl | 396.4 | 1.68 |
| 63 | 3-thienyl | 3-methoxy-phenyl | 401.0 | 1.68 |
| 64 | 3-thienyl | 2-bromo-phenyl | 449.2 | 1.86 |
| 65 | 3-thienyl | 3-bromo-phenyl | 449.2 | 1.86 |
| 66 | 3-thienyl | 4-bromo-phenyl | 449.2 | 1.91 |
| 67 | 3-thienyl | hexyl | 377.0 | 1.92 |
| 68 | phenyl | 2-fluoro-phenyl | 383.2 | 1.79 |
| 69 | phenyl | 3-fluoro-phenyl | 383.2 | 1.77 |
| 70 | phenyl | 4-cyano-phenyl | 39.2 | 1.70 |
| 71 | phenyl | 3-methoxy-phenyl | 395.4 | 1.79 |
| 72 | phenyl | 2-bromo-phenyl | 443.2 | 1.87 |
| 73 | phenyl | 3-bromo-phenyl | 443.4 | 1.91 |
| 74 | phenyl | 4-bromo-phenyl | 443.2 | 1.89 |
| 75 | phenyl | 4-methoxy-phenyl | 395.4 | 1.76 |
| 76 | phenyl | phenyl | 365.6 | 1.70 |
| 77 | 2-thienyl | phenyl | 371.2 | 1.77 |
| 78 | 2-thienyl | 2-fluoro-phenyl | 389.4 | 1.72 |
| 79 | 2-thienyl | 3-fluoro-phenyl | 389.4 | 1.74 |
| 80 | 2-thienyl | 4-fluoro-phenyl | 389.2 | 1.77 |
| 81 | 2-thienyl | 2,4-difluorophenyl | 407.4 | 1.81 |
| 82 | 2-thienyl | 2-bromo-phenyl | 449.2 | 1.84 |
| 83 | 2-thienyl | 3-bromo-phenyl | 449.2 | 1.87 |
| 84 | 2-thienyl | 4-bromo-phenyl | 449.2 | 1.90 |
| 85 | 2-thienyl | 4-methoxy-phenyl | 401.0 | 1.72 |

### Example 86

### 3-(endo)-8-azabicyclo[3.2.1]oct-3-ylmethyl (2-fluoro-5-methylphenyl)(phenylmethyl)carbamate trifluoroacetate

### 86-a Preparation of 2-fluoro-5-methyl-N-(phenylmethyl)aniline

Following the standard procedure outlined in Example 22a, 2-fluoro-5-methylaniline (500 mg, 4 mmol) was reacted with benzaldehyde (473 mg, 4.5 mmol) in the presence of NaB(OAc)₃H (3.6 g, 16.8 mmol) and acetic acid (680 mg, 11.3 mmol) to give 2-fluoro-5-methyl-N-(phenylmethyl)aniline (500 mg). LC/MS: m/z, 216 (M+H).

### 86-b Preparation of the Title Compound

Following the standard procedure outlined in Example 22b, 1,1-dimethylethyl-(3-endo)-3-(hydroxymethyl)-8-azabicyclo[3.2.1]octane-8-carboxylate (72.3 mg, 0.3 mmol) was reacted with 2-fluoro-5-methyl-N-(phenylmethyl)aniline (64.5 mg, 0.3 mmol) in the presence of triphosgene (29.7 mg, 0.1 mmol) to give the title compound (50.4 mg). LC/MS: m/z, 383 (M+H), 1.75 min.

### Example 87

### (3-endo)-3-[({[(2-fluoro-5-methylphenyl)(phenylmethyl)amino]carbonyl}oxy)methyl]-8,8-dimethyl-8-azoniabicyclo[3.2.1]octane iodide

Following the standard procedure outlined in Example 2, (3-endo)-8-azabicyclo[3.2.1]oct-3-ylmethyl (2-fluoro-5-methylphenyl)(phenylmethyl)carbamate trifluoroacetate (50 mg, 0.13 mmol) was reacted with iodomethane (185 mg, 1.3 mmol) and Na₂CO₃ (69 mg, 0.65 mmol) to give the title compound (58.2 mg, 83%). LC/MS: m/z, 367 (M+H), 1.85 min.

### Example 88

### (3-endo)-8-azabicyclo[3.2.1]oct-3-ylmethyl (3-chlorophenyl)(phenylmethyl)carbamate trifluoroacetate

### 88-a Preparation of 3-chloro-N-(phenylmethyl)aniline

Following the standard procedure outlined in Example 22a, 3-chloroaniline (472 mg, 3.70 mmol) was reacted with benzaldehyde (473 mg, 4.5 mmol) in the presence of NaB(OAc)₃H (3.32 g, 15.6 mmol) and acetic acid (0.6 ml, 10.5 mmol) to give 3-chloro-N-(phenylmethyl)aniline (690 mg, 85%). LC/MS: m/z, 218 (M+H), 2.43 min.

### 88-b Preparation of the Title Compound

Following the standard procedure outlined in Example 22b, (3-endo)-1,1-dimethylethyl-3-(hydroxymethyl)-8-azabicyclo[3.2.1]octane-8-carboxylate (750 mg, 3.1 mmol) was reacted with 3-chloro-N-(phenylmethyl)aniline (218 mg, 1.0 mmol) in the presence of triphosgene (360 mg, 1.24 mmol) to give the title compound (120 mg, 37%). LC/MS: m/z, 385 (M+H), 1.68 min.

### Example 89

### (3-endo)-8-azabicyclo[3.2.1]oct-3-ylmethyl[(3-hydroxyphenyl)methyl]2-thienylcarbamate hydrobromide

A solution of (3-endo)-8-azabicyclo[3.2.1]oct-3-ylmethyl{[3-(methyloxy)phenyl]methyl}2-thienylcarbamate (40 mg, 0.082 mmol) in DCM (1 ml) was mixed with 0.2 ml of 1M of B-bromo-9-borabicyclo[3.3.1]nonanein (B—Br-9-BBN) in DCM. The solution was heated at reflux for 12 hours then quenched with methanol (1 ml). The cooled solution was evaporated under vacuum to give a crude yellow oil, which was further purified by Gilson HPLC, eluting with acetonitrile/water/O. 1% TFA (10/90 to 90/10, v/v, over 10 min), to give the title compound (18 mg, 50%). LC/MS: m/z, 373 (M+H), 1.43 min.

### Example 90

### (3-endo)-8-azabicyclo[3.2.1]oct-3-ylmethyl[(3,5-dihydroxyphenyl)methyl]2-thienyl carbamate hydrobromide

Following the standard procedure outlined in Example 89, (3-endo)-8-azabicyclo[3.2.1]oct-3-ylmethyl{[3,5-bis(methyloxy)phenyl]methyl}2-thienylcarbamate (186 mg, 0.36 mmol) was reacted with 2 ml of a 1M solution of B—Br-9-BBN (2 mmol) in DCM to give the title compound (100 mg, 60%). LC/MS: m/z, 389(M+H), 1.84 min.

### Example 91

### (3-endo)-8-azabicyclo[3.2.1]oct-3-ylmethyl (3-hydroxyphenyl)[(3-hydroxyphenyl)methyl]carbamate hydrobromide

### 91-a Preparation of 3-endo-1,1-dimethylethyl-3-{[({[3-(methyloxy)phenyl]amino}carbonyl)oxy]methyl}-8-azabicyclo[3.2.1]octane-8-carboxylate

Following the standard procedure outlined for Intermediate 1,3-(methyloxy)benzoic acid (1.52 g, 10 mmol) was reacted with (3-endo)-1,1-dimethylethyl 3-(hydroxymethyl)-8-azabicyclo[3.2.1]octane-8-carboxylate (3.62 mg, 15 mmol) and diphenylphosphoryl azide (3.0 g, 11 mmol) to afford 3-endo-1,1-dimethylethyl-3-{[({[3-(methyloxy)phenyl]amino}carbonyl)oxy]methyl}-8-azabicyclo[3.2.1]octane-8-carboxylate (3.18 g). LC/MS: m/z, 391 (M+H), 2.46 min.

### 91-b Preparation of (3-endo)-1,1-dimethylethyl-3-({[([3-(methyloxy)phenyl]{[3-(methyloxy)phenyl]methyl}amino)carbonyl]oxy}methyl)-8-azabicyclo[3.2.1]octane-8-carboxylate

Following the standard procedure outlined for Intermediate 2, (3-endo)-1,1-dimethylethyl-3-{[({[3-(methyloxy)phenyl]amino}carbonyl)oxy]methyl}-8-azabicyclo[3.2.1]octane-8-carboxylate (200 mg, 0.51 mmol) was reacted with 1-(bromomethyl)-3-(methyloxy)benzene (206 mg, 1.02 mmol) to give (3-endo)-1,1-dimethylethyl-3-({[([3-(methyloxy)phenyl]{[3-(methyloxy)phenyl]methyl}amino)carbonyl]oxy}methyl)-8-azabicyclo[3.2.1]octane-8-carboxylate (240 mg). LC/MS: m/z, 511 (M+H), 2.73 min.

### 91-c Preparation of the Title Compound

Following the standard procedure outlined in Example 89, (3-endo)-1,1-dimethylethyl-3-({[([3-(methyloxy)phenyl]{[3-(methyloxy)phenyl]methyl}amino)carbonyl]oxy}methyl)-8-azabicyclo[3.2.1]octane-8-carboxylate (240 mg, 0.47 mmol) was reacted with 2.6 ml of 1M B—Br-9-BBN (2.6 mmol) to give the title compound (205 mg, 94%). LC/MS: m/z, 383 (M+H), 1.40 min.

### Example 92

### (3-endo)-8-azabicyclo[3.2.1]oct-3-ylmethyl[(4-cyanophenyl)methyl](2-hydroxyphenyl)carbamate hydrobromide

### 92-a Preparation of (3-endo)-1,1-dimethylethyl-3-{[({[2-(methyloxy)phenyl]amino}carbonyl)oxy]methyl}-8-azabicyclo[3.2.1]octane-8-carboxylate

Following the standard procedure outlined for intermediate 1,2-(methyloxy)benzoic acid (1.52 g, 10 mmol) was reacted with (3-endo)-1,1-dimethylethyl 3-(hydroxymethyl)-8-azabicyclo[3.2.1]octane-8-carboxylate (3.6 mg, 15 mmol) and diphenylphosphoryl azide (3.0 g, 11 mmol) to give (3-endo)-1,1-dimethylethyl-3-{[({[2-(methyloxy)phenyl]amino}carbonyl)oxy]methyl}-8-azabicyclo[3.2.1]octane-8-carboxylate (3.1 g). LC/MS: m/z, 391 (M+H), 2.59 min.

### 92-b Preparation of (3-endo)-1,1-dimethylethyl-3-{[({[(4-cyanophenyl)methyl][2-(methyloxy)phenyl]amino}carbonyl)oxy]methyl}-8-azabicyclo[3.2.1]octane-8-carboxylate

Following the standard procedure outlined for intermediate 2, (3-endo)-1,1-dimethylethyl-3-{[({[2-(methyloxy)phenyl]amino}carbonyl)oxy]methyl}-8-azabicyclo[3.2.1]octane-8-carboxylate (200 mg, 0.512 mmol) was reacted with 3-(bromomethyl)benzonitrile (196 mg, 1.02 mmol) to give 3-endo)-1,1-dimethylethyl-3-{[({[(4-cyanophenyl)methyl][2-(methyloxy)phenyl]amino}carbonyl)oxy]methyl}-8-azabicyclo[3.2.1]octane-8-carboxylate (250 mg). LC/MS: m/z, 506 (M+H).

### 92-c Preparation of the Title Compound

Following the standard procedure outlined in Example 89, (3-endo)-1,1-dimethylethyl-3-{[({[(4-cyanophenyl)methyl][2-(methyloxy)phenyl]amino}carbonyl)oxy]methyl}-8-azabicyclo[3.2.1]octane-8-carboxylate (240 mg, 0.47 mmol) was reacted with 2.6 ml of a solution of IM B—Br-9-BBN (2.6 mmol) in DCM to give the title compound (180 mg, 93%). LC/MS: m/z, 392 (M+H), 1.74 min.

### Example 93

### (3-endo)-8-azabicyclo[3.2.1]oct-3-ylmethyl (2-hydroxyphenyl)[(3-hydroxyphenyl)methyl]carbamate hydrobromide

### 93-a Preparation of (3-endo)-1,1-dimethylethyl-3-({[([2-(methyloxy)phenyl]{[3-(methyloxy)phenyl]methyl}amino)carbonyl]oxy}methyl)-8-azabicyclo[3.2.1]octane-8-carboxylate

Following the standard procedure outlined for intermediate 1, (3-endo)-1,1-dimethylethyl-3-{[({[2-(methyloxy)phenyl]amino}carbonyl)oxy]methyl}-8-azabicyclo[3.2.1]octane-8-carboxylate (200 mg, 0.512 mmol) was reacted with 1-(bromomethyl)-3-(methyloxy)benzene (196 mg, 1.02 mmol) to give (3-endo)-1,1-dimethylethyl-3-({[([2-(methyloxy)phenyl]{[3-(methyloxy)phenyl]methyl}amino)carbonyl]oxy}methyl)-8-azabicyclo[3.2.1]octane-8-carboxylate (240 mg). LC/MS: m/z, 511 (M+H), 2.88 min.

### 93-b Preparation of the Title Compound

Following the standard procedure outlined in Example 89, (3-endo)-1,1-dimethylethyl-3-({[([2-(methyloxy)phenyl]{[3-(methyloxy)phenyl]methyl}amino)carbonyl]oxy}methyl)-8-azabicyclo[3.2.1]octane-8-carboxylate (240 mg, 0.47 mmol) was reacted with 2.6 ml of a 1M solution of B—Br-9-BBN in DCM (2.6 mmol) to give the title compound (172 mg, 96%). LC/MS: m/z, 383 (M+H), 1.64 min.

### Example 94

### (3-endo)-8-methyl-8-azabicyclo[3.2.1]oct-3-yl cyclopentyl[(4-fluorophenyl)methyl]carbamate

Cyclopentylamine (42.6 mg, 0.5 mmol) and MP-cyanoborohydride (from Argonaut Technologies Inc., PN 800406) (400 mg, 1.0 mmol) were added sequentially added to a solution of 4-fluorobenzaldehyde (53 μl, 0.5 mmol) in THF (3 ml) and acetic acid (1 ml). After stirring at room temperature under argon for 66 hours, the reaction mixture was filtered off and the resin washed with THF (2 ml). The combined filtrates were evaporated under vacuum to give a crude material which was partitioned between DCM (5 ml) and 2.5 M aq. NaOH (2 ml). The organic phase was separated using a hydrophobic frit and the aqueous layer was further extracted with DCM (2 ml). The organic layers were combined to afford the desired secondary amine in DCM (as assessed by LC/MS). To the solution thus prepared, triethylamine (0.11 ml, 1.11 mmol) and ethylchloroformate (0.33 ml, 3.5 mmol) were sequentially added. The resulting mixture was stirred at room temperature for 96 hours. More DCM (2 ml) was added and the mixture was loaded onto a 2 g silica SPE cartridge then eluted with DCM (10 ml). After evaporation of the solvent under vacuum a crude material containing the corresponding ethyl carbamate was recovered as an oil residue.

A solution of the commercially available (3-endo)-8-methyl-8-azabicyclo[3.2.1]octan-3-ol (also known as tropine, Aldrich cat. # T8-940-0) (144.2 mg, 1.0 mmol) in toluene (1.0 ml) was treated with sodium hydride (25 mg, 1.0 mmol) under argon for 10 minutes at 0° C. To this mixture was added the crude ethyl carbamate previously prepared dissolved in THF (1 ml). The reaction mixture was heated at 140° C. in a microwave (Emrys Optimizer) for 50 minutes then quenched with 2 droplets of water. The solvent was evaporated under vacuum to give a residue which was partitioned between DCM (5 ml) and water (3 ml). The organic phase was separated using a hydrophobic frit and the aqueous layer washed with DCM (3 ml). The organic layers were combined, loaded onto a 2 g aminopropyl SPE cartridge and sequentially eluted with DCM (20 ml), ethyl acetate (10 ml), ethyl acetate:methanol 9:1 (10 ml) and methanol (10 ml). The ethyl acetate fractions were combined and evaporated to give the title compound (55.2 mg). LCMS: m/z, 361 (M+H), 1.76 min.

The compounds listed in Table 3 were prepared proceeding in a similar manner to Example 94, but replacing 4-fluorobenzaldehyde with the appropriate substituted benzaldehyde.

TABLE 3

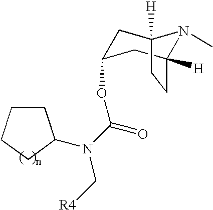

| Example | n | R4 | MS [M+] | Rt (min) |
|---|---|---|---|---|
| 95 | 1 | 2-bromo-phenyl | 421.2 | 1.95 |
| 96 | 1 | 2-methoxy-phenyl | 373.3 | 1.86 |
| 97 | 1 | 3-fluoro-phenyl | 360.6 | 1.76 |
| 98 | 1 | phenyl | 343 | 1.71 |
| 99 | 1 | 4-bromo-phenyl | 421.2 | 1.92 |
| 100 | 1 | 3-methoxy-phenyl | 373.2 | 1.73 |

### Example 101

### (3-endo)-3-[({cyclopentyl[(4-fluorophenyl)methyl]amino}carbonyl)oxy]-8,8-dimethyl-8-azoniabicyclo[3.2.1]octane bromide

Methyl bromide (0.43 ml of a 2.0 M in tert-butyl methyl ether, 0.86 mmol) was added to a solution of (3-endo)-8-methyl-8-azabicyclo[3.2.1]oct-3-yl cyclopentyl[(4-fluorophenyl)methyl]carbamate (34.3 mg, 0.095 mmol) in DCM (1 ml) and acetonitrile (1 ml). The resulting mixture was stirred at room temperature for 3 hours then evaporated under vacuum to give the title compound as a white solid (43 mg). LC/MS m/z, 375.5 (M+), 1.94 min.

The compounds listed in Table 4 were prepared proceeding in a similar manner to Example 101, but replacing (3-endo)-8-methyl-8-azabicyclo[3.2.1]oct-3-yl cyclopentyl[(4-fluorophenyl)methyl]carbamate with the appropriate tertiary amine.

TABLE 4

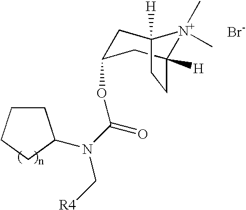

| Example | n | R4 | MS [M+] | Rt (min) |
|---|---|---|---|---|
| 103 | 1 | 3-bromo-phenyl | 435.2 | 2.01 |
| 104 | 1 | 3-fluoro-phenyl | 375.0 | 1.86 |
| 105 | 1 | phenyl | 357.2 | 1.77 |

### Example 106

### (3-endo)-3-[({cyclohexyl[(2-fluorophenyl)methyl]amino}carbonyl)oxy]-8,8-dimethyl-8-azoniabicyclo[3.2.1]octane bromide

Cyclohexylamine (57 μl, 0.5 mmol) and MP-cyanoborohydride (from Argonaut Technologies Inc., PN 800406) (400 mg, 1.0 mmol) were added sequentially added to a solution of 2-fluorobenzaldehyde (53 μl, 0.5 mmol) in THF (3 ml) and acetic acid (1 ml). After stirring at room temperature under argon for 17 hours, the reaction mixture was filtered off and the resin washed with THF (2 ml). The combined filtrates were evaporated under vacuum to give a crude material which was partitioned between DCM (5 ml) and 2.5 M aq. NaOH (2 ml). The organic phase was separated using a hydrophobic frit and the aqueous layer was further extracted with DCM (2 ml). The organic layers were combined to afford the desired secondary amine in DCM (as assessed by LC/MS). To the solution thus prepared, triethylamine (0.17 ml, 1.25 mmol) and ethylchloroformate (0.33 ml, 3.5 mmol) were sequentially added. The resulting mixture was stirred at room temperature for 21 hours then loaded onto a 2 g silica SPE cartridge then eluted with DCM (12 ml). After evaporation of the solvent under vacuum a crude material containing the corresponding ethyl carbamate was recovered as an oil residue.

A solution of the commercially available (3-endo)-8-methyl-8-azabicyclo[3.2.1]octan-3-ol (also known as tropine, Aldrich cat. # T8-940-0) (144.2 mg, 1.0 mmol) in toluene (1.0 ml) was treated with sodium hydride (25 mg, 1.0 mmol) under argon for 30 minutes at 0° C. To this mixture was added the crude ethyl carbamate previously prepared dissolved in THF (2 ml). The reaction mixture was heated at 140° C. in a microwave (Emrys Optimizer) for 50 minutes then quenched with 2 droplets of water. The solvent was evaporated under vacuum to give a residue which was partitioned between DCM (4 ml) and water (4 ml). The organic phase was separated using a hydrophobic frit and the aqueous layer washed with DCM (2 ml). The organic layers were combined, loaded onto a 2 g aminopropyl SPE cartridge and sequentially eluted with hexane (10 ml), DCM (10 ml), diethyl ether (10 ml), ethyl acetate (15 ml), ethyl acetate:methanol 9:1 (10 ml) and methanol (10 ml). The fractions containing the desired product as assessed by TLC, were combined and evaporated to give crude (3-endo)-8-methyl-8-azabicyclo[3.2.1]oct-3-yl cyclohexyl[(2-fluorophenyl)methyl]carbamate. To this material dissolved in DCM (1 ml) and acetonitrile (1 ml) was added methyl bromide (0.5 ml of a 2.0 M in tert-butyl methyl ether, 1.0 mmol). The resulting mixture was stirred at room temperature for 3 hours then evaporated under vacuum to give the title compound as a white solid (37 mg). LC/MS m/z, 389.2 (M+), 1.91 min.

The compounds listed in Table 5 were prepared proceeding in a similar manner to Example 106, but replacing 2-fluorobenzaldehyde with the appropriate substituted benzaldehyde and cyclohexylamine with the appropriate substituted cycloalkylamine.

TABLE 5

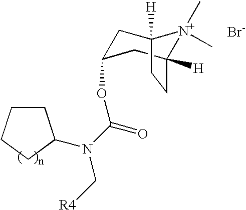

| Example | n | R4 | MS [M+] | Rt (min) |
|---|---|---|---|---|
| 102 | 1 | 2-fluoro-phenyl | 375.2 | 1.90 |
| 107 | 2 | 4-fluoro-phenyl | 391.0 | 1.97 |
| 108 | 2 | 3-bromo-phenyl | 449.2 | 2.09 |
| 109 | 3 | 4-fluoro-phenyl | 403.2 | 2.07 |

### Example 110

### (3-endo)-8-methyl-8-azabicyclo[3.2.1]oct-3-yl phenyl(2-thienylmethyl)carbamate

### 110-a: Preparation of (2-thienylmethyl)phenylamine

To a solution of aniline (47 mg, 0.5 mmol) and 2-thiophenecarboxaldehyde (56 mg, 0.5 mmol) in THF (3 ml) and acetic acid (1 ml), was added 400 mg of MP-cyanoborohydride resin (Argonaut, 2.57 mmol/g). The mixture was stirred at room temperature for 18 h. After filtration, the filtrate was evaporated and diluted in DCM (5 ml) and 2N aq. NaOH (2 ml). The organic layer was separated using a hydrophobic frit. Removal of the solvent yielded a crude oil, which was directly used for the next step.

### 110-b: Preparation of ethyl (2-thienylmethyl)phenylcarbamate

The above crude was dissolved in of anhydrous THF (5 ml). To this solution was added triethylamine (127 mg, 1.5 mmol) and ethyl chloroformate (380 mg, 3.5 mmol) at 0° C. The mixture was stirred at room temperature for 18 hours. The solvent was removed and the residue was diluted in DCM (5 ml) and 2N aq. NaOH (2 ml). The organic layer was separated using a hydrophobic frit. Removal of the solvent yielded a crude material, which was used directly for the next step.

### 110-c: Preparation of the Title Compound

To a suspension of NaH (28 mg, 1.2 mmol) in anhydrous toluene (1 ml), was added a solution of (3-endo)-8-methyl-8-azabicyclo[3.2.1]octan-3-ol (a.k.a. tropine, 1.0 mmol) in dry toluene (1.5 ml) at 0° C. The mixture was stirred for 30 min, then mixed with the crude residue previously isolated in step b. The solution was heated at reflux for 1 h, and cooled to room temperature. One drop of water was added to quench the reaction. The reaction mixture washed with water (1 ml) and evaporated under vacuum to give a crude product, which was purified using an aminopropyl solid-phase extraction cartridge (Applied Separations, 2 g) and eluting sequentially with DCM, ethyl acetate then methanol. The ethyl acetate fraction was evaporated to give the title compound (53 mg). LC/MS: m/z, 357, 1.63 min.

The compounds listed in Table 6 were prepared proceeding in a similar manner to Example 110, but replacing 2-thiophenecarboxaldehyde with the appropriate aldehyde.

TABLE 6

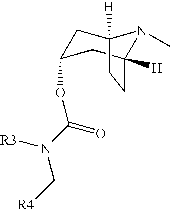

| Example | R3 | R4 | MS [M+] | Rt (min) |
|---|---|---|---|---|
| 111 | phenyl | 3-thienyl | 357 | 1.54 |
| 112 | phenyl | 3-methyl-2-thienyl | 371 | 1.60 |
| 113 | phenyl | 5-methyl-2-thienyl | 371 | 1.57 |
| 114 | phenyl | 3-furyl | 341 | 1.48 |
| 115 | phenyl | 5-methyl-2-furyl | 355 | 1.51 |
| 116 | phenyl | 2-furyl | 341 | 1.55 |

### Example 117

### (3-endo)-8,8-dimethyl-3-({[phenyl(2-thienylmethyl)amino]carbonyl}oxy)-8-azoniabicyclo[3.2.1]octane bromide

To a solution of (3-endo)-8-methyl-8-azabicyclo[3.2.1]oct-3-yl phenyl(2-thienylmethyl)carbamate (50 mg, 0.14 mmol) in acetonitrile/DCM (1:1 v/v) (1 ml) was added methyl bromide (1.4 ml of a 2.0 M in tert-butyl methyl ether, 2.8 mmol). The resulting mixture was stirred at room temperature for 60 hours. The title compound was obtained by filtration and washing with dry ether (29 mg, 46%). LC/MS: m/z, 372, 1.64 min.

The compounds listed in Table 7 were prepared proceeding in a similar manner to Example 117, but replacing (3-endo)-8-methyl-8-azabicyclo[3.2.1]oct-3-yl phenyl(2-thienylmethyl)carbamate with the appropriate tertiary amine.

TABLE 7

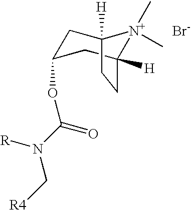

| Example | R3 | R4 | MS [M+] | Rt (min) |
|---|---|---|---|---|
| 118 | phenyl | 3-thienyl | 372 | 1.55 |
| 119 | phenyl | 3-methyl-2-thienyl | 385 | 1.70 |
| 120 | phenyl | 5-methyl-2-thienyl | 385 | 1.64 |
| 121 | phenyl | 3-furyl | 355 | 1.48 |
| 122 | phenyl | 5-methyl-2-furyl | 369 | 1.55 |
| 123 | phenyl | 2-furyl | 355 | 1.52 |

### Example 124

### (3-endo)-8-methyl-8-azabicyclo[3.2.1]oct-3-yl 3-thienyl(3-thienylmethyl)carbamate

The title compound was prepared proceeding in a similar manner to Example 32, but replacing (4-fluorophenyl)methyl bromide with 3-bromomethyl thiophene (prepared according to a literature method:. J. Chem. Soc., Perkin Trans., I, 1999, 2639-2644). Yield: 25 mg (18%). LC/MS: m/z, 363, 1.64 min.

### Example 125

### (3-endo)-8,8-dimethyl-3-({[3-thienyl(3-thienylmethyl)amino]carbonyl}oxy)-8-azoniabicyclo[3.2.1]octane bromide

To a solution of (3-endo)-8-methyl-8-azabicyclo[3.2.1]oct-3-yl 3-thienyl(3-thienylmethyl)carbamate (20 mg, 0.055 mmol) in acetone (1 ml) was added methyl bromide (0.55 ml of a 2.0 M in tert-butyl methyl ether, 1.1 mmol). The mixture was stirred at room temperature for 16 hours. The title compound was obtained by filtration and washing with dry ether (24 mg, 96%). LC/MS: m/z, 377, 1.66 min.

### Example 126

### (3-endo)-8-azabicyclo[3.2.1]oct-3-yl 3-thienyl(3-thienylmethyl)carbamate

Following the standard procedure outlined in Example 1, 1,1-dimethylethyl (3-endo)-3-{[(3-thienylamino)carbonyl]oxy}-8-azabicyclo[3.2.1]octane-8-carboxylate (Intermediate 4, 1.0 g, 2.84 mmol) was reacted with 3-bromomethyl thiophene (603 mg, 3.4 mmol prepared according to a literature method. J. Chem. Soc., Perkin Trans., 1, 1999, 2639-2644) to give the title compound (920 mg, 93%). LC/MS: m/z, 349, 1.36 mm.

### Example 127

### (3-endo)-8-azabicyclo[3.2.1]oct-3-yl 2-thienyl(3-thienylmethyl)carbamate

Following the standard procedure outlined in Example 1, 1,1-dimethylethyl (3-endo)-3-{[(2-thienylamino)carbonyl]oxy}-8-azabicyclo[3.2.1]octane-8-carboxylate (Intermediate 5, 200 m g, 0.568 mmol) was reacted with 3-bromomethyl thiophene (109 mg, 0.625 mmol, prepared according to a literature method. J. Chem. Soc., Perkin Trans., I, 1999, 2639-2644) to give the title compound (160 mg, 81%). LC/MS: m/z, 349, 1.49 min.

### Example 128

### (3-endo)-8,8-dimethyl-3-({[2-thienyl(3-thienylmethyl)amino]carbonyl}oxy)-8-azoniabicyclo[3.2.1]octane iodide

A solution of (3-endo)-8-azabicyclo[3.2.1]oct-3-yl 2-thienyl(3-thienylmethyl)carbamate (50 mg, 0.144 mmol) was dissolved in methanol (1 ml) to which was added methyl iodide (2.88 mmol) and K₂CO₃ (199 mg, 1.44 mmol). The mixture was stirred at room temperature for 16 hours. The title compound was obtained by filtration and washing with dry ether (57 mg, 78%). LC/MS: m/z, 377, 1.66 mm.

### Example 129

### (3-endo)-8-(phenylmethyl)-8-azabicyclo[3.2.1]oct-3-yl 3-thienyl(3-thienylethyl)carbamate

To a solution of (3-endo)-8-azabicyclo[3.2.1]oct-3-yl 3-thienyl(3-thienylmethyl)carbamate (50 mg, 0.144 mmol) in anhydrous acetonitrile (2 ml), was added bromomethyl benzene (29 mg, 0.17 mmol) and K₂CO₃ (117 mg, 0.85 mmol). The mixture was heated at reflux for 3 hours. After cooling to room temperature, the solution was filtered off, and the filtrate was concentrated under vacuum to yield a crude material, which was further purified using an aminopropyl SPE cartridge (Applied Separations, 2 g). Yield: 47 mg (76%). LC/MS: m/z, 439, 1.97 min.

The compounds listed in Table 8 were prepared proceeding in a similar manner to Example 129, but replacing bromomethyl benzene with the appropriate alkyl halide.

TABLE 8

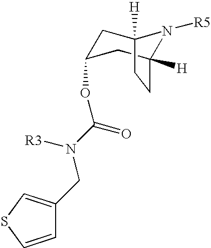

| Example | R3 | R5 | MS [M+] | Rt (min) |
|---|---|---|---|---|
| 130 | 3-thienyl | (3-phenyloxy)propyl | 483 | 1.97 |
| 131 | 3-thienyl | propyl | 392 | 1.68 |
| 132 | 3-thienyl | 2-propen-1-yl | 389 | 1.78 |
| 133 | 3-thienyl | 3-cyanopropyl | 416 | 1.69 |
| 134 | 3-thienyl | 5-hexen-1-yl | 431 | 1.94 |
| 135 | 3-thienyl | 2-{[2-methloxy)ethyl] | 451 | 1.64 |
| | | oxy}ethyl | | |
| 136 | 3-thienyl | 6-hydroxy-1-hexyl | 449 | 1.74 |
| 137 | 3-thienyl | 2-phenylethyl | 453 | 1.91 |
| 138 | 3-thienyl | 3-phenylpropyl | 467 | 1.94 |
| 139 | 3-thienyl | 4-[(phenylmethyl)oxyy]- | 511 | 2.00 |
| | | 1-butyl | | |
| 140 | 2-thienyl | propyl | 391 | 1.81 |
| 141 | 2-thienyl | 3-cyanopropyl | 416 | 1.52 |
| 142 | 2-thienyl | 5-hexen-1-yl | 431 | 1.96 |
| 143 | 2-thienyl | 2-{[2-methloxy)ethyl] | 451 | 1.68 |
| | | oxy}ethyl | | |
| 144 | 2-thienyl | 6-hydroxy-1-hexyl | 449 | 1.70 |
| 145 | 2-thienyl | benzyl | 439 | 2.00 |
| 146 | 2-thienyl | 2-phenylethyl | 453 | 1.83 |
| 147 | 2-thienyl | 3-phenylpropyl | 467 | 1.93 |
| 148 | 2-thienyl | 4-[(phenylmethyl)oxyy]- | 511 | 1.94 |
| | | 1-butyl | | |
| 149 | 2-thienyl | (3-phenyloxy)propyl | 483 | 1.91 |
| 150 | 2-thienyl | 4-penten-1-yl | 417 | 1.73 |
| 151 | 2-thienyl | cyclohexylmethyl | 445 | 2.04 |

### Example 152

### (3-endo)-8-methyl-8-[3-(phenyloxy)propyl]-3-({[3-thienyl(3-thienylmethyl)amino]carbonyl}oxy)-8-azoniabicyclo[3.2.1]octane bromide

To a solution of (3-endo)-8-[3-(phenyloxy)propyl]-8-azabicyclo[3.2.1]oct-3-yl 3-thienyl(3-thienylmethyl)carbamate (30 mg, 0.062 mmol) in acetonitrile/DCM (1:1 v/v) (1 ml) was added methyl bromide (0.62 ml of a 2.0 M in tert-butyl methyl ether, 1.24 mmol). The mixture was stirred at room temperature for 16 hours. The title compound was obtained by filtration and washing with dry ether (31 mg, 74%). LC/MS: m/z, 497, 2.03 min.

The compounds listed in Table 9 were prepared proceeding in a similar manner to Example 152, but replacing (3-endo)-8-[3-(phenyloxy)propyl]-8-azabicyclo[3.2.1]oct-3-yl-3-thienyl(3-thienylmethyl)carbamate with the appropriate tertiary amine.

TABLE 9

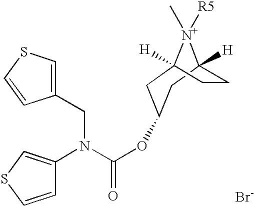

| Example | R5 | MS [M+] | Rt (min) |
|---|---|---|---|
| 153 | propyl | 406 | 1.82 |
| 154 | 5-hexen-1-yl | 445 | 1.81 |
| 155 | 2-{[2-methloxy)ethyl]oxy}ethyl | 465 | 1.76 |
| 156 | 6-hydroxy-1-hexyl | 463 | 1.64 |
| 157 | 2-phenylethyl | 467 | 1.80 |
| 158 | 3-phenylpropyl | 482 | 1.90 |
| 159 | 4-[(phenylmethyl)oxyy]-1-butyl | 525 | 1.88 |
| 160 | benzyl | 453 | 1.91 |

### Example 161

### (3-endo)-8-methyl-8-propyl-3-({[2-thienyl(3-thienylmethyl)amino]carbonyl}oxy)-8-azoniabicyclo[3.2.1]octane iodide

To a solution of (3-endo)-8-propyl-8-azabicyclo[3.2.1]oct-3-yl 2-thienyl(3-thienylmethyl)carbamate (15 mg, 0.038 mmol) in acetonitrile/DCM (1:1 v/v) (1 ml) was added methyl iodide (0.76 mmol). The mixture was stirred at room temperature for 16 hours. The title compound was obtained by filtration and washing with dry ether (15 mg, 74%).

The compounds listed in Table 10 were prepared proceeding in a similar manner to Example 161, but replacing (3-endo)-8-propyl-8-azabicyclo[3.2.1]oct-3-yl 2-thienyl(3-thienylmethyl)carbamate with the appropriate tertiary amine.

TABLE 10

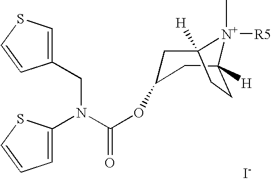

| Example | R5 | MS [M+] | Rt (min) |
|---|---|---|---|
| 162 | 5-hexen-1-yl | 445 | 1.86 |
| 163 | 2-{[2-methloxy)ethyl]oxy}ethyl | 465 | 1.86 |
| 164 | benzyl | 453 | 1.71 |
| 165 | 2-phenylethyl | 467 | 1.87 |
| 166 | 3-phenylpropyl | 481 | 1.84 |
| 167 | 4-[(phenylmethyl)oxyy]-1-butyl | 525 | 1.94 |
| 168 | 3-(phenyloxy)propyl | 498 | 1.93 |
| 169 | 4-penten-1-yl | 432 | 1.68 |

Abbreviations
A.k.a Also known as
B—Br-9-BBN B-Bromo-9-borabicyclo[3.3.1]nonane
BOC tert-butyloxycarbonyl
DCM Dichloromethane
DMF Dimethylformamide
DMSO Dimethylsulfoxide
ESI Electrospray ionization
HPLC High pressure liquid chromatography
LC/MS Liquid chromatography/mass spectrometry
NMR Nuclear magnetic resonance
SPE Solid phase extraction
TEA Triethylamine
TFA Trifluoroacetic acid
THF Tetrahydrofuran
TLC Thin layer chromatography

### BIOLOGICAL EXAMPLES

The inhibitory effects of compounds at the M₃ mAChR of the present invention are determined by the following in vitro and in vivo functional assays:

Analysis of Inhibition of Receptor Activation by Calcium Mobilization:

Stimulation of mAChRs expressed on CHO cells were analyzed by monitoring receptor-activated calcium mobilization as previously described (H. M. Sarau et al, 1999. Mol. Pharmacol. 56, 657-663). CHO cells stably expressing M₃ mAChRs were plated in 96 well black wall/clear bottom plates. After 18 to 24 hours, media was aspirated and replaced with 100 μl of load media (EMEM with Earl's salts, 0.1% RIA-grade BSA (Sigma, St. Louis Mo.), and 4 μM Fluo-3-acetoxymethyl ester fluorescent indicator dye (Fluo-3 AM, Molecular Probes, Eugene, Oreg.) and incubated 1 hr at 37° C. The dye-containing media was then aspirated, replaced with fresh media (without Fluo-3 AM), and cells were incubated for 10 minutes at 37° C. Cells were then washed 3 times and incubated for 10 minutes at 37° C. in 100 μl of assay buffer (0.1% gelatin (Sigma), 120 mM NaCl, 4.6 mM KCl, 1 mM KH₂ PO₄, 25 mM NaH CO₃, 1.0 mM CaCl₂, 1.1 mM MgCl₂, 11 mM glucose, 20 mM HEPES (pH 7.4)). 50 μl of compound (1×10⁻¹¹-1×10⁻⁵ M final in the assay) was added and the plates were incubated for 10 min. at 37° C. Plates were then placed into a fluorescent light intensity plate reader (FLIPR, Molecular Probes) where the dye loaded cells were exposed to excitation light (488 nm) from a 6 watt argon laser. Cells were activated by adding 50 μl of acetylcholine (0.1-10 nM final), prepared in buffer containing 0.1% BSA, at a rate of 50 μl/sec. Calcium mobilization, monitored as change in cytosolic calcium concentration, was measured as change in 566 nm emission intensity. The change in emission intensity is directly related to cytosolic calcium levels. The emitted fluorescence from all 96 wells is measured simultaneously using a cooled CCD camera. Data points are collected every second. This data was then plotting and analyzed using GraphPad PRISM software.

Methacholine-Induced Bronchoconstriction—Potency and Duration of Action

Airway responsiveness to methacholine was determined in awake, unrestrained Balb C mice (n=6 each group). Barometric plethysmography was used to measure enhanced pause (Penh), a unitless measure that has been shown to correlate with the changes in airway resistance that occur during bronchial challenge with methacholine(2). Mice were pre-treated with 50 μl of compound (0.003-10 μg/mouse) in 50 μl of vehicle (10% DMSO) intranasally (i.n.) and were then placed in the plethysmography chamber a given amount of time following drug administration (15 min—96 h). For potency determination, a dose response to a given drug was performed, and all measurements were taken 15 min following i.n. drug administration. For duration of action determination, measurements were taken anywhere from 15 min to 96 hours following i.n. drug administration.

Once in the chamber, the mice were allowed to equilibrate for 10 min before taking a baseline Penh measurement for 5 minutes. Mice were then challenged with an aerosol of methacholine (10 mg/ml) for 2 minutes. Penh was recorded continuously for 7 min starting at the inception of the methacholine aerosol, and continuing for 5 minutes afterward. Data for each mouse were analyzed and plotted by using GraphPad PRISM software. This experiment allows the determination of duration of activity of the administered compound.

The present compounds are useful for treating a variety of indications, including but not limited to respiratory-tract disorders such as chronic obstructive lung disease, chronic bronchitis, asthma, chronic respiratory obstruction, pulmonary fibrosis, pulmonary emphysema, and allergic rhinitis.

### Formulation

### Administration

Accordingly, the present invention further provides a pharmaceutical formulation comprising a compound of formula (I), or a pharmaceutically acceptable salt, solvate, or physiologically functional derivative (e.g., salts and esters) thereof, and a pharmaceutically acceptable carrier or excipient, and optionally one or more other therapeutic ingredients.

Hereinafter, the term “active ingredient” means a compound of formula (I), or a pharmaceutically acceptable salt, solvate, or physiologically functional derivative thereof.

Compounds of formula (I) will be administered via inhalation via the mouth or nose.

Dry powder compositions for topical delivery to the lung by inhalation may, for example, be presented in capsules and cartridges of for example gelatine, or blisters of for example laminated aluminium foil, for use in an inhaler or insufflator. Powder blend formulations generally contain a powder mix for inhalation of the compound of the invention and a suitable powder base (carrier/diluent/excipient substance) such as mono-, di- or poly-saccharides (e.g., lactose or starch), organic or inorganic salts (e.g., calcium chloride, calcium phosphate or sodium chloride), polyalcohols (e.g., mannitol), or mixtures thereof, alternatively with one or more additional materials, such additives included in the blend formulation to improve chemical and/or physical stability or performance of the formulation, as discussed below, or mixtures thereof. Use of lactose is preferred. Each capsule or cartridge may generally contain between 20 μg-10 mg of the compound of formula (I) optionally in combination with another therapeutically active ingredient. Alternatively, the compound of the invention may be presented without excipients, or may be formed into particles comprising the compound, optionally other therapeutically active materials, and excipient materials, such as by co-precipitation or coating.

Suitably, the medicament dispenser is of a type selected from the group consisting of a reservoir dry powder inhaler (RDPI), a multi-dose dry powder inhaler (MDPI), and a metered dose inhaler (MDI).

By reservoir dry powder inhaler (RDPI) it is meant as an inhaler having a reservoir form pack suitable for comprising multiple (un-metered doses) of medicament in dry powder form and including means for metering medicament dose from the reservoir to a delivery position. The metering means may for example comprise a metering cup or perforated plate, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

The formulation can be pre-metered (eg as in Diskus, see GB 2242134 or Diskhaler, see GB 2178965, 2129691 and 2169265) or metered in use (eg as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one aspect, the multi-dose pack is a blister pack comprising multiple blisters for containment of medicament in dry powder form. The blisters are typically arranged in regular fashion for ease of release of medicament therefrom.

In one aspect, the multi-dose blister pack comprises plural blisters arranged in generally circular fashion on a disk-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the compound of formula (I) optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants eg oleic acid or lecithin and cosolvents eg ethanol. Pressurized formulations will generally be retained in a canister (eg an aluminium canister) closed with a valve (eg a metering valve) and fitted into an actuator provided with a mouthpiece.

Medicaments for administration by inhalation desirably have a controlled particle size. The optimum aerodynamic particle size for inhalation into the bronchial system for localized delivery to the lung is usually 1-10 μm, preferably 2-5 μm. The optimum aerodynamic particle size for inhalation into the alveolar region for achieving systemic delivery to the lung is approximately 0.5-3 μm, preferably 1-3 μm. Particles having an aerodynamic size above 20 μm are generally too large when inhaled to reach the small airways. Average aerodynamic particle size of a formulation may measured by, for example cascade impaction. Average geometric particle size may be measured, for example by laser diffraction, optical means.

To achieve a desired particle size, the particles of the active ingredient as produced may be size reduced by conventional means eg by controlled crystallization, micronisation or nanomilling. The desired fraction may be separated out by air classification. Alternatively, particles of the desired size may be directly produced, for example by spray drying, controlling the spray drying parameters to generate particles of the desired size range. Preferably, the particles will be crystalline, although amorphous material may also be employed where desirable. When an excipient such as lactose is employed, generally, the particle size of the excipient will be much greater than the inhaled medicament within the present invention, such that the “coarse” carrier is non-respirable. When the excipient is lactose it will typically be present as milled lactose, wherein not more than 85% of lactose particles will have a MMD of 60-90 μm and not less than 15% will have a MMD of less than 15 μm. Additive materials in a dry powder blend in addition to the carrier may be either respirable, i.e., aerodynamically less than 10 microns, or non-respirable, i.e., aerodynamically greater than 10 microns.

Suitable additive materials which may be employed include amino acids, such as leucine; water soluble or water insoluble, natural or synthetic surfactants, such as lecithin (e.g., soya lecithin) and solid state fatty acids (e.g., lauric, palmitic, and stearic acids) and derivatives thereof (such as salts and esters); phosphatidylcholines; sugar esters. Additive materials may also include colorants, taste masking agents (e.g., saccharine), anti-static-agents, lubricants (see, for example, Published PCT Patent Appl. No. WO 87/905213, the teachings of which are incorporated by reference herein), chemical stabilizers, buffers, preservatives, absorption enhancers, and other materials known to those of ordinary skill.

Sustained release coating materials (e.g., stearic acid or polymers, e.g. polyvinyl pyrolidone, polylactic acid) may also be employed on active material or active material containing particles (see, for example, Patent Nos. U.S. Pat. No. 3,634,582, GB 1,230,087, GB 1,381,872, the teachings of which are incorporated by reference herein).

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Preferred unit dosage formulations are those containing an effective dose, as herein before recited, or an appropriate fraction thereof, of the active ingredient.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

What is claimed is:

1. A compound according to Formula (I) represented by the structure: 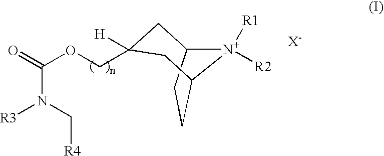
wherein:
R1 is a bond, hydrogen or C1-4 alkyl;
R2 is selected from the group consisting of hydrogen, C1-10 alkyl, halosubstitued C1-10 alkyl, C1-10 alkyl cyano, C2-10 alkenyl, cycloalkyl, C1-10 alkylcycloalkyl, cycloakyl C1-10 alkyl, and (CR8R8)q-ORa;
R3 and R4 are independently selected from the group consisting of 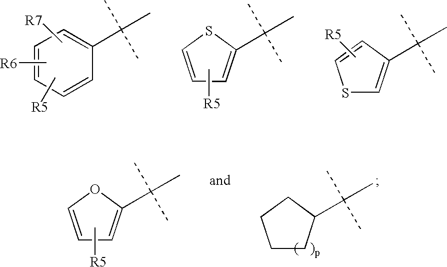
wherein, R5, R6 and R7 are, independently, selected from the group consisting of hydrogen, halogen, C1-4 alkyl, C2-5 alkenyl, C1-4 alkoxy, halosubstituted C1-4 alkoxy, halosubstituted C1-4 alkyl, hydroxy, and cyano;
n is an integer having a value of 0 to 2;
p is an integer having a value of 0 to 3;
q is an integer having a value of 2 to 10;
Ra is selected from the group consisting of hydrogen, C1-10 alkyl, aryl, aryl C1-10 alkyl, C1-4 alkyl aryl, halosubstituted C1-10 alkyl, C1-10 alkoxy, halosubstituted C1-10 alkoxy, C1-10 alkyl cyano and C2-10 alkenyl;
R8 is hydrogen, halogen or C1-4 alkyl; and
X— is a physiologically acceptable anion.

2. A compound according to claim 1 of formula (I) herein below: 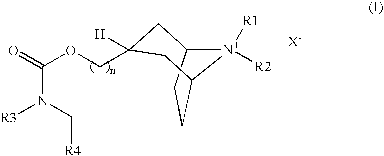
wherein:
R1 is a bond, hydrogen or C1-4 alkyl
R2 is selected from the group consisting of hydrogen, C1-4 alkyl, C2-5 alkenyl, C1-4 alkylcycloalkyl, and (CR8R8)q-ORa;
R3 and R4 are independently selected from the group consisting of 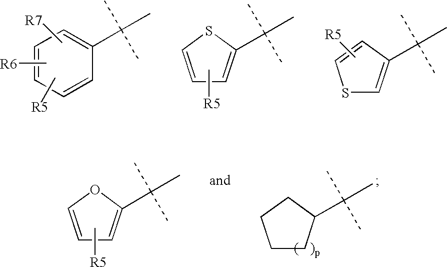
wherein R5, R6 and R7, are independently, selected from the group consisting of hydrogen, halogen, C1-4 alkyl, C2-5 alkenyl, C1-4 alkoxy, and cyano;
n is an integer having a value of 0 or 1;
p is an integer having a value of 1 or 2;
q is an integer having a value of 2 to 4;
Ra is selected from the group consisting of hydrogen, C1-4 alkyl, aryl, aryl C1-4 alkyl, C1-4 alkyl aryl, and C1-10 alkyl;
R8 is hydrogen; and
X— is selected from the group consisting of chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate.

3. A compound according to claim 1 selected from the group of:
(3-endo)-8,8-dimethyl-3-({[3-thienyl(3-thienylmethyl)amino]carbonyl}oxy)-8-azoniabicyclo[3.2.1]octane bromide;
(3-endo)-8,8-dimethyl-3-({[(phenylmethyl)(3-thienyl)amino]carbonyl}oxy)-8-azoniabicyclo[3.2.1]octane bromide;
(3-endo)-3-({[[(3-fluorophenyl)methyl](3-thienyl)amino]carbonyl}oxy)-8,8-dimethyl-8-azoniabicyclo[3.2.1]octane bromide;
(3-endo)-8,8-dimethyl-3-({[phenyl(2-thienylmethyl)amino]carbonyl}oxy)-8-azoniabicyclo[3.2.1]octane bromide;
(3-endo)-3-({[[(4-fluorophenyl)methyl](3-thienyl)amino]carbonyl}oxy)-8,8-dimethyl-8-azoniabicyclo[3.2.1]octane bromide;
(3-endo)-8,8-dimethyl-3-({[phenyl(3-thienylmethyl)amino]carbonyl}oxy)-8-azoniabicyclo[3.2.1]octane bromide;
(3-endo)-8,8-dimethyl-3-({[(phenylmethyl)(2-thienyl)amino]carbonyl}oxy)-8-azoniabicyclo[3.2.1]octane bromide;
(3-endo)-3-({[[(4-fluorophenyl)methyl](2-thienyl)amino]carbonyl}oxy)-8,8-dimethyl-8-azoniabicyclo[3.2.1]octane bromide;
(3-endo)-3-({[[(2-fluorophenyl)methyl](3-thienyl)amino]carbonyl}oxy)-8,8-dimethyl-8-azoniabicyclo[3.2.1]octane bromide;
(3-endo)-3-({[[(3-fluorophenyl)methyl](2-thienyl)amino]carbonyl}oxy)-8,8-dimethyl-8-azoniabicyclo[3.2.1]octane bromide;
(3-endo)-3-({[[(3-fluorophenyl)methyl](phenyl)amino]carbonyl}oxy)-8,8-dimethyl-8-azoniabicyclo[3.2.1]octane bromide;
(3-endo)-3-({[[(2-fluorophenyl)methyl](2-thienyl)amino]carbonyl}oxy)-8,8-dimethyl-8-azoniabicyclo[3.2.1]octane bromide;
(3-endo)-8,8-dimethyl-3-({[phenyl(phenylmethyl)amino]carbonyl}oxy)-8-azoniabicyclo[3.2.1]octane bromide;
(3-endo)-3-({[[(2,4-difluorophenyl)methyl](2-thienyl)amino]carbonyl}oxy)-8,8-dimethyl-8-azoniabicyclo[3.2.1]octane bromide;
(3-endo)-8,8-dimethyl-3-({[2-thienyl(3-thienylmethyl)amino]carbonyl}oxy)-8-azoniabicyclo[3.2.1]octane iodide;
(3-endo)-8-azabicyclo[3.2.1]oct-3-yl 2-thienyl(3-thienylmethyl)carbamate trifluoroacetate;
(3-endo)-8-azabicyclo[3.2.1]oct-3-yl 3-thienyl(3-thienylmethyl)carbamate trifluoroacetate;
(3-endo)-8,8-dimethyl-3-({[[(5-methyl-2-thienyl)methyl](phenyl)amino]carbonyl}oxy)-8-azoniabicyclo[3.2.1]octane bromide;
(3-endo)-8-azabicyclo[3.2.1]oct-3-ylmethyl (phenylmethyl)2-thienylcarbamate;
(3-endo)-8-methyl-8-azabicyclo[3.2.1]oct-3-yl 3-thienyl(3-thienylmethyl)carbamate;
(3-endo)-8-methyl-8-azabicyclo[3.2.1]oct-3-yl [(4-fluorophenyl)methyl]3-thienylcarbamate;
(3-endo)-8-methyl-8-azabicyclo[3.2.1]oct-3-yl (phenylmethyl)3-thienylcarbamate;
(3-endo)-3-({[(cyclohexylmethyl)(3-thienyl)amino]carbonyl}oxy)-8,8-dimethyl-8-azoniabicyclo[3.2.1]octane bromide;
(3-endo)-8-azabicyclo[3.2.1]oct-3-ylmethyl[(4-fluorophenyl)methyl]2-thienylcarbamate;
(3-endo)-8-azabicyclo[3.2.1]oct-3-ylmethyl[(3,4-difluorophenyl)methyl]2-thienylcarbamate;
(3-endo)-8-(6-hydroxyhexyl)-8-methyl-3-({[3-thienyl(3-thienylmethyl)amino]carbonyl}oxy)-8-azoniabicyclo[3.2.1]octane bromide;
(3-endo)-8-azabicyclo[3.2.1]oct-3-ylmethyl[(2-fluorophenyl)methyl]3-thienylcarbamate;
(3-endo)-3-({[[(2-fluorophenyl)methyl](phenyl)amino]carbonyl}oxy)-8,8-dimethyl-8-azoniabicyclo[3.2.1]octane bromide;
(3-endo)-8-azabicyclo[3.2.1]oct-3-ylmethyl[(3-fluorophenyl)methyl]2-thienylcarbamate;
(3-endo)-8-azabicyclo[3.2.1]oct-3-ylmethyl[(3-fluorophenyl)methyl]3-thienylcarbamate;
(3-endo)-8-azabicyclo[3.2.1]oct-3-ylmethyl[(3,5-difluorophenyl)methyl]3-thienylcarbamate;
(3-endo)-8-azabicyclo[3.2.1]oct-3-ylmethyl 3-thienyl[(2,4,5-trifluorophenyl)methyl]carbamate;
(3-endo)-8-methyl-8-azabicyclo[3.2.1]oct-3-yl [(3-fluorophenyl)methyl]3-thienylcarbamate;
(3-endo)-8-azabicyclo[3.2.1]oct-3-ylmethyl[(2-fluorophenyl)methyl]2-thienylcarbamate;
(3-endo)-3-({[(3-furanylmethyl)(phenyl)amino]carbonyl}oxy)-8,8-dimethyl-8-azoniabicyclo[3.2.1]octane bromide;
(3-endo)-8-azabicyclo[3.2.1]oct-3-ylmethyl[(2,4-difluorophenyl)methyl]2-thienylcarbamate;
(3-endo)-8-azabicyclo[3.2.1]oct-3-ylmethyl[(2,3-difluorophenyl)methyl]3-thienylcarbamate;
(3-endo)-8-azabicyclo[3.2.1]oct-3-ylmethyl[(2,5-difluorophenyl)methyl]2-thienylcarbamate;
(3-endo)-8-azabicyclo[3.2.1]oct-3-ylmethyl phenyl(phenylmethyl)carbamate;
(3-endo)-8-methyl-8-azabicyclo[3.2.1]oct-3-yl phenyl(2-thienylmethyl)carbamate;
(3-endo)-8-methyl-8-azabicyclo[3.2.1]oct-3-yl [(2-fluorophenyl)methyl]3-thienylcarbamate;
(3-endo)-8,8-dimethyl-3-({[[(3-methyl-2-thienyl)methyl](phenyl)amino]carbonyl}oxy)-8-azoniabicyclo[3.2.1]octane bromide;
(3-endo)-8-azabicyclo[3.2.1]oct-3-ylmethyl 2-thienyl[(2,3,4-trifluorophenyl)methyl]carbamate;
(3-e do)-8-methyl-8-azabicyclo[3.2.1]oct-3-yl (phenylmethyl)2-thienylcarbamate;
(3-endo)-8-methyl-8-azabicyclo[3.2.1]oct-3-yl [(3-fluorophenyl)methyl]2-thienylcarbamate;
(3-endo)-8-methyl-8-azabicyclo[3.2.1]oct-3-yl phenyl(3-thienylmethyl)carbamate;
(3-endo)-8-methyl-8-azabicyclo[3.2.1]oct-3-yl [(4-fluorophenyl)methyl]2-thienylcarbamate;
(3-endo)-8-(6-hydroxyhexyl)-8-azabicyclo[3.2.1]oct-3-yl 3-thienyl(3-thienylmethyl)carbamate;
(3-endo)-8-methyl-8-azabicyclo[3.2.1]oct-3-yl [(2-fluorophenyl)methyl]2-thienylcarbamate;
(3-endo)-3-({[[(4-bromophenyl)methyl](3-thienyl)amino]carbonyl}oxy)-8,8-dimethyl-8-azoniabicyclo[3.2.1]octane bromide; and
(3-endo)-8,8-dimethyl-3-({[[(5-methyl-2-furanyl)methyl](phenyl)amino]carbonyl}oxy)-8-azoniabicyclo[3.2.1]octane bromide.

4. A compound according to claim 3 selected from the group consisting of:
(3-endo)-8,8-dimethyl-3-({[3-thienyl(3-thienylmethyl)amino]carbonyl}oxy)-8-azoniabicyclo[3.2.1]octane bromide;
(3-endo)-8,8-dimethyl-3-({[(phenylmethyl)(3-thienyl)amino]carbonyl}oxy)-8-azoniabicyclo[3.2.1]octane bromide;
(3-endo)-3-({[[(3-fluorophenyl)methyl](3-thienyl)amino]carbonyl}oxy)-8,8-dimethyl-8-azoniabicyclo[3.2.1]octane bromide;
(3-endo)-8,8-dimethyl-3-({[phenyl(2-thienylmethyl)amino]carbonyl}oxy)-8-azoniabicyclo[3.2.1]octane bromide;
(3-endo)-3-({[[(4-fluorophenyl)methyl](3-thienyl)amino]carbonyl}oxy)-8,8-dimethyl-8-azoniabicyclo[3.2.1]octane bromide;
(3-endo)-8,8-dimethyl-3-({[phenyl(3-thienylmethyl)amino]carbonyl}oxy)-8-azoniabicyclo[3.2.1]octane bromide;
(3-endo)-8,8-dimethyl-3-({[(phenylmethyl)(2-thienyl)amino]carbonyl}oxy)-8-azoniabicyclo[3.2.1]octane bromide;
(3-endo)-3-({[[(4-fluorophenyl)methyl](2-thienyl)amino]carbonyl}oxy)-8,8-dimethyl-8-azoniabicyclo[3.2.1]octane bromide;
(3-endo)-3-({[[(2-fluorophenyl)methyl](3-thienyl)amino]carbonyl}oxy)-8,8-dimethyl-8-azoniabicyclo[3.2.1]octane bromide;
(3-endo)-3-({[[(3-fluorophenyl)methyl](2-thienyl)amino]carbonyl}oxy)-8,8-dimethyl-8-azoniabicyclo[3.2.1]octane bromide;
(3-endo)-3-({[[(3-fluorophenyl)methyl](phenyl)amino]carbonyl}oxy)-8,8-dimethyl-8-azoniabicyclo[3.2.1]octane bromide;
(3-endo)-3-({[[(2-fluorophenyl)methyl](2-thienyl)amino]carbonyl}oxy)-8,8-dimethyl-8-azoniabicyclo[3.2.1]octane bromide;
(3-endo)-8,8-dimethyl-3-({[phenyl(phenylmethyl)amino]carbonyl}oxy)-8-azoniabicyclo[3.2.1]octane bromide;
(3-endo)-3-({[[(2,4-difluorophenyl)methyl](2-thienyl)amino]carbonyl}oxy)-8,8-dimethyl-8-azoniabicyclo[3.2.1]octane bromide;
(3-endo)-8,8-dimethyl-3-({[2-thienyl(3-thienylmethyl)amino]carbonyl}oxy)-8-azoniabicyclo[3.2.1]octane iodide;
(3-endo)-8-azabicyclo[3.2.1]oct-3-yl 2-thienyl(3-thienylmethyl)carbamate trifluoroacetate; and
(3-endo)-8-azabicyclo[3.2.1]oct-3-yl 3-thienyl(3-thienylmethyl)carbamate trifluoroacetate.

5. A pharmaceutical composition for the treatment of muscarinic acetylcholine receptor mediated diseases comprising a compound according to claim 1 and a pharmaceutically acceptable carrier thereof.

6. A method of inhibiting the binding of acetylcholine to its receptors in a mammal in need thereof comprising administering a safe and effective amount of a compound according to claim 1.

7. A method of treating a muscarinic acetylcholine receptor mediated disease, wherein acetylcholine binds to said receptor, comprising administering a safe and effective amount of a compound according to claim 1.

8. A method according to claim 7 wherein the disease is selected from the group consisting of chronic obstructive lung disease, chronic bronchitis, asthma, chronic respiratory obstruction, pulmonary fibrosis, pulmonary emphysema and allergic rhinitis.

9. A method according to claim 7 wherein administration is via inhalation via the mouth or nose.

10. A method according to claim 7 wherein administration is via a medicament dispenser selected from a reservoir dry powder inhaler, a multi-dose dry powder inhaler or a metered dose inhaler.
